(19) United States
(12) Patent Application Publication
(10) Pub. No.: US 2009/0286775 A1
(43) Pub. Date: Nov. 19, 2009

(54) Bicyclic Derivatives as P38 Kinase Inhibitors

(75) Inventors: Carmen Almansa Rosales, Barcelona (ES); Marina Virgili Bernadó, Barcelona (ES)

Correspondence Address: ROTHWELL, FIGG, ERNST & MANBECK, P.C., 1425 K STREET, N.W., SUITE 800, WASHINGTON, DC, 20005, US

(73) Assignee: PALAU PHARMA, S.A., Palau-solità i Plegamans (ES)

(21) Appl. No.: 11/993,261

(22) PCT Filed: Jun. 28, 2006
(86) PCT No.: PCT/EP2006/006255

§ 371 Date: Dec. 20, 2007

(30) Foreign Application Priority Data
Jun. 29, 2005 (EP) 05380140.3

(51) Int. Cl. A61K 31/4035; C07D 209/44; C07D 417/02; C07D 213/00; C07D 265/30; C07D 401/02; A61K 31/427; A61K 31/44; A61K 31/5375; A61K 31/4439; A61P 37/00; A61P 29/00; A61P 9/00; A61P 25/00

(52) U.S. Cl. 514/231.2; 548/470; 548/181; 546/329; 544/162; 546/277.1; 514/416; 514/365; 514/357; 514/339

(57) ABSTRACT

New bicyclic derivatives of formula (I), wherein the meanings for the various substituents are as disclosed in the description. These compounds are useful as p38 kinase inhibitors.

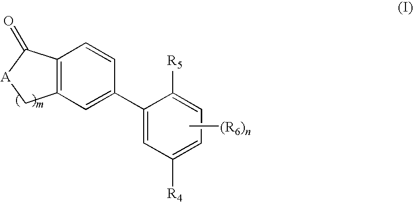

### FIELD OF THE INVENTION

The present invention relates to a new series of bicyclic derivatives, to processes to prepare them, to pharmaceutical compositions comprising these compounds as well as to their use in therapy.

### BACKGROUND OF THE INVENTION

Kinases are proteins involved in different cellular responses to external signals. In the Nineties, a new family of kinases called MAPK (mitogen-activated protein kinases) was discovered. MAPK activate their substrates by phosphorylation in serine and threonine residues.

MAPK are activated by other kinases in response to a wide range of signals including growth factors, pro-inflammatory cytokines, UV radiation, endotoxins and osmotic stress. Once they are activated, MAPK activate by phosphorylation other kinases or proteins, such as transcription factors, which, ultimately, induce an increase or a decrease in expression of a specific gene or group of genes.

The MAPK family includes kinases such as p38, ERK (extracellular-regulated protein kinase) and JNK (C-Jun N-terminal kinase).

p38 kinase plays a crucial role in cellular response to stress and in the activation pathway in the synthesis of numerous cytokines, especially tumor necrosis factor (TNF-α), interleukin-1 (IL-1), interleukin-6 (IL-6) and interleukin-8 (IL-8).

IL-1 and TNF-α are produced by macrophages and monocytes and are involved in the mediation of immunoregulation processes and other physiopathological conditions. For example, elevated levels of TNF-α are associated with inflammatory and autoimmune diseases and with processes that trigger the degradation of connective and bone tissue such as rheumatoid arthritis, osteoarthritis, diabetes, inflammatory bowel disease and sepsis.

Thus, it is believed that p38 kinase inhibitors can be useful to treat or prevent diseases mediated by cytokines such as IL-1 and TNF-α, such as the ones mentioned above.

On the other hand, it has also been found that p38 inhibitors inhibit other pro-inflammatory proteins such as IL-6, IL-8, interferon-γ and GM-CSF (granulocyte-macrophage colony-stimulating factor). Moreover, in recent studies it has been found that p38 inhibitors not only block cytokine synthesis but also the cascade of signals that these induce, such as induction of the cyclooxygenase-2 enzyme (COX-2).

Accordingly, it would be desirable to provide novel compounds which are capable of inhibiting the p38 kinase.

WO 2004/108672 discloses compounds containing a isoindolin-1-one moiety as inhibitors of certain protein tyrosine kinases, particularly KDR.

### DESCRIPTION OF THE INVENTION

One aspect of the present invention relates to the compounds of general formula I

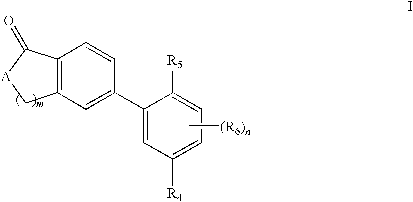

wherein:
A represents CR₁R₂ or NR₃;
R₁ and R₂ independently represent C₁₋₄ alkyl;
R₃ represents —(CH₂)ₚ—Cy¹, or C₁₋₆ alkyl optionally substituted with one or more R₇;
m represents 1 or 2;
R₄ represents —B—R₈;
R₅ represents hydrogen, C₁₋₄ alkyl, halogen or C₁₋₄ alkoxy;
R₆ can be attached to any available carbon atom of the phenyl ring and represents halogen or methyl;
n represents 0 or 1;
B represents —CONR₉—, —NR₉CO— or —NR₉CONR₉—;
R₇ represents hydroxy, C₁₋₄ alkoxy, halogen, —NR₁₀R₁₀ or phenyl optionally substituted with one or more groups selected from C₁₋₄ alkyl, halogen, C₁₋₄ alkoxy, C₁₋₄ haloalkyl and C₁₋₄ haloalkoxy, and additionally two R₇ groups on the same carbon atom can be bonded together to form a —(CH₂)_{q}— group;
R₈ represents C₁₋₆ alkyl or —(CH₂)ₚ—Cy²;
p represents 0, 1 or 2;
q represents 2, 3, 4, 5 or 6;
Cy¹ represents phenyl, heteroaryl, C₃₋₇ cycloalkyl or heterocyclyl, which can all be optionally substituted with one or more R₁₁;
Cy² represents phenyl, heteroaryl or C₃₋₇ cycloalkyl, which can all be optionally substituted with one or more R₁₂;
R₉ and R₁₀ independently represent hydrogen or C₁₋₄ alkyl;
R₁₁ represents halogen, R₁₃, —OR₁₃, —NO₂, —CN, —COR_{13′}, —CO₂R_{13′}, —CONR_{14′}R_{14′}, —NR_{14′}R_{14′}, —NR_{14′}COR_{13′}, —NR_{14′}CONR_{14′}R_{14′}, —NR_{14′}CO₂R₁₃, —NR_{14′}SO₂R₁₃, —SR_{13′}, —SOR₁₃, —SO₂R₁₃, —SO₂NR_{14′}R_{14′}, or Cy³;
R₁₂ represents C₁₋₄ alkyl, halogen, C₁₋₄ alkoxy, C₁₋₄ haloalkyl, C₁₋₄ haloalkoxy, or Cy³;
R₁₃ represents C₁₋₄ alkyl, C₁₋₄ haloalkyl or C₁₋₄ hydroxyalkyl;
R_{13′} represents hydrogen or R₁₃;
R₁₄ represents C₁₋₄ alkyl or C₁₋₄ hydroxyalkyl;
R_{14′} represents hydrogen or R₁₄; and
Cy³ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl and C₁₋₄ haloalkoxy.

The present invention also relates to the salts and solvates of the compounds of formula I.

Some compounds of formula I can have chiral centres that can give rise to various stereoisomers. The present invention relates to each of these stereoisomers and also mixtures thereof.

The compounds of formula I are p38 kinase inhibitors and also inhibit the production of cytokines such as TNF-α.

Thus, another aspect of the invention relates to a compound of general formula I

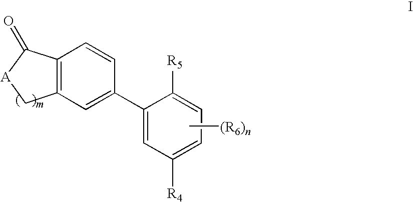

wherein:
A represents CR₁R₂ or NR₃;
R₁ and R₂ independently represent C₁₋₄ alkyl;
R₃ represents —(CH₂)ₚ—Cy¹, or C₁₋₆ alkyl optionally substituted with one or more R₇;
m represents 1 or 2;
R₄ represents —B—R₈;
R₅ represents hydrogen, C₁₋₄ alkyl, halogen or C₁₋₄ alkoxy;
R₆ can be attached to any available carbon atom of the phenyl ring and represents halogen or methyl;
n represents 0 or 1;
B represents —CONR₉—, —NR₉CO— or —NR₉CONR₉—;
R₇ represents hydroxy, C₁₋₄ alkoxy, halogen, —NR₁₀R₁₀ or phenyl optionally substituted with one or more groups selected from C₁₋₄ alkyl, halogen, C₁₋₄ alkoxy, C₁₋₄ haloalkyl and C₁₋₄ haloalkoxy, and additionally two R₇ groups on the same carbon atom can be bonded together to form a —(CH₂)_{q}— group;
R₈ represents C₁₋₆ alkyl or —(CH₂)ₚ—Cy²;
p represents 0, 1 or 2;
q represents 2, 3, 4, 5 or 6;
Cy¹ represents phenyl, heteroaryl, C₃₋₇ cycloalkyl or heterocyclyl, which can all be optionally substituted with one or more R₁₁;
Cy² represents phenyl, heteroaryl or C₃₋₇ cycloalkyl, which can all be optionally substituted with one or more R₁₂;
R₉ and R₁₀ independently represent hydrogen or C₁₋₄ alkyl;
R₁₁ represents halogen, R₁₃, —OR_{13′}, —NO₂, —CN, —COR_{13′}, —CO₂R_{13′}, —CONR_{14′}R_{14′}, —NR_{14′}R_{14′}, —NR_{14′}COR_{13′}, —NR_{14′}CONR_{14′}R_{14′}, —NR_{14′}CO₂R₁₃, —NR_{14′}SO₂R₁₃, —SR_{13′}, —SOR₁₃, —SO₂R₁₃, —SO₂NR_{14′}R_{14′}, or Cy³;
R₁₂ represents C₁₋₄ alkyl, halogen, C₁₋₄ alkoxy, C₁₋₄ haloalkyl, C₁₋₄ haloalkoxy, or Cy³;
R₁₃ represents C₁₋₄ alkyl, C₁₋₄ haloalkyl or C₁₋₄ hydroxyalkyl;
R_{13′} represents hydrogen or R₁₃;
R₁₄ represents C₁₋₄ alkyl or C₁₋₄ hydroxyalkyl;
R_{14′} represents hydrogen or R₁₄; and
Cy³ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl and C₁₋₄ haloalkoxy, for use in therapy.

Another aspect of this invention relates to a pharmaceutical composition which comprises a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment or prevention of diseases mediated by p38.

Another aspect of the present invention relates to the use of a compound of formula I or a pharmaceutically acceptable salt thereof for the manufacture of a medicament for the treatment or prevention of diseases mediated by cytokines.

Another aspect of the present invention relates to the use of a compound of formula I or a pharmaceutically acceptable salt thereof for the manufacture of a medicament for the treatment or prevention of diseases mediated by TNF-α, IL-1, IL-6 and/or IL-8.

Another aspect of the present invention relates to the use of a compound of formula I or a pharmaceutically acceptable salt thereof for the manufacture of a medicament for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to the use of a compound of formula I or a pharmaceutically acceptable salt thereof for the treatment or prevention of diseases mediated by p38.

Another aspect of the present invention relates to the use of a compound of formula I or a pharmaceutically acceptable salt thereof for the treatment or prevention of diseases mediated by cytokines.

Another aspect of the present invention relates to the use of a compound of formula I or a pharmaceutically acceptable salt thereof for the treatment or prevention of diseases mediated by TNF-α, IL-1, IL-6 and/or IL-8.

Another aspect of the present invention relates to the use of a compound of formula I or a pharmaceutically acceptable salt thereof for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to a method of treating or preventing a disease mediated by p38 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by cytokines in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by TNF-α, IL-1, IL-6 and/or IL-8 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease selected from immune, autoimmune and inflammatory diseases, cardiovascular diseases, infectious diseases, bone resorption disorders, neurodegenerative diseases, proliferative diseases and processes associated with the induction of cyclooxygenase-2 in a subject in need thereof, especially a human being, which comprises administering to said subject a therapeutically effective amount of a compound of formula I or a pharmaceutically acceptable salt thereof.

Another aspect of the present invention relates to a process for the preparation of a compound of formula I as defined above, which comprises:

(a) reacting a compound of formula II with a compound of formula III

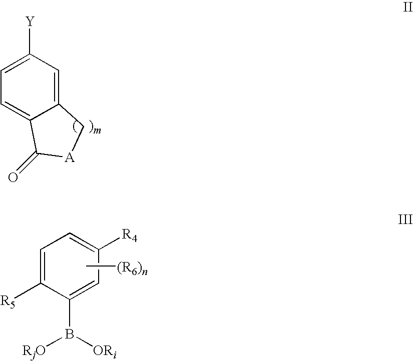

wherein A, R₄, R₅, R₆, m and n have the meaning described above, Y represents halogen or trifluoromethanesulfonate, and each Rᵢ and Rⱼ represent H or C₁₋₄alkyl or they can be linked together to form together with the B and O atoms a five or six-membered ring which can be optionally substituted by one or more methyl groups; or
(b) when in a compound of formula I R₄ represents —CONR₉R₈, reacting a compound of formula IV with an amine of formula HNR₈R₉ (V)

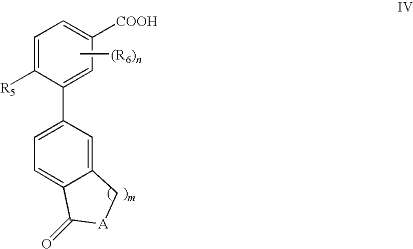

wherein A, R₅, R₆, R₈, R₉, m and n have the meaning described above; or
(c) when in a compound of formula I R₄ represents —NHCOR₈, reacting a compound of formula VI with an acid of formula R₈COOH (VII)

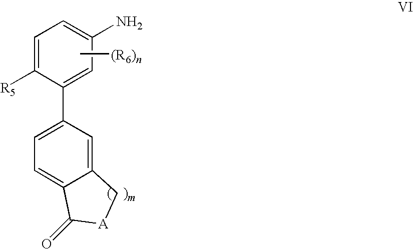

wherein A, R₅, R₆, R₈, m and n have the meaning described above; or
(d) when in a compound of formula I R₄ represents —NHCONHR₈, reacting a compound of formula VI with an isocyanate of formula R₈NCO (VIII); or
(e) converting, in one or a plurality of steps, a compound of formula I into another compound of formula I.

In the above definitions, the term C₁₋ₙ alkyl, as a group or part of a group, means a straight or branched alkyl chain which contains from 1 to n carbon atoms. When n is 4, it includes the groups methyl, ethyl, propyl, isopropyl, butyl, isobutyl, sec-butyl and tert-butyl. When n is 6, examples include among others the groups methyl, ethyl, propyl, isopropyl, butyl, isobutyl, sec-butyl, tert-butyl, pentyl, isopentyl, neopentyl and hexyl.

A C₁₋₄ haloalkyl group means a group resulting from the replacement of one or more hydrogen atoms from a C₁₋₄alkyl group with one or more halogen atoms (i.e. fluoro, chloro, bromo or iodo), which can be the same or different. Examples include, among others, trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl and nonafluorobutyl.

A C₁₋₄ alkoxy group means an alkoxy group having from 1 to 4 carbon atoms, the alkyl moiety having the same meaning as previously defined. Examples include methoxy, ethoxy, propoxy, isopropoxy, butoxy, isobutoxy, sec-butoxy and tert-butoxy.

A C₁₋₄ haloalkoxy group means a group resulting from the replacement of one or more hydrogen atoms from a C₁₋₄ alkoxy group with one or more halogen atoms (i.e. fluoro, chloro, bromo or iodo), which can be the same or different. Examples include, among others, trifluoromethoxy, fluoromethoxy, 1-chloroethoxy, 2-chloroethoxy, 1-fluoroethoxy, 2-fluoroethoxy, 2-bromoethoxy, 2-iodoethoxy, 2,2,2-trifluoroethoxy, pentafluoroethoxy, 3-fluoropropoxy, 3-chloropropoxy, 2,2,3,3-tetrafluoropropoxy, 2,2,3,3,3-pentafluoropropoxy, heptafluoropropoxy, 4-fluorobutoxy and nonafluorobutoxy.

A C₁₋₄ hydroxyalkyl group means a group resulting from the replacement of one or more hydrogen atoms from a C₁₋₄ alkyl group with one or more hydroxy groups. Examples include, among others, hydroxymethyl, 1-hydroxyethyl, 2-hydroxyethyl, 1,2-dihydroxyethyl, 3-hydroxypropyl, 2-hydroxypropyl, 1-hydroxypropyl, 2,3-dihydroxypropyl, 4-hydroxybutyl, 3-hydroxybutyl, 2-hydroxybutyl and 1-hydroxybutyl.

A halogen radical means fluoro, chloro, bromo or iodo.

A C₃₋₇ cycloalkyl group means a saturated monocyclic hydrocarbon ring having 3 to 7 carbon atoms, i.e. cyclopropyl, cyclobutyl, cyclopentyl, cyclohexyl and cycloheptyl.

The term heteroaryl means an aromatic 5- or 6-membered monocyclic or 8- to 12-membered bicyclic ring which contains from 1 to 4 heteroatoms selected from N, S and O. The heteroaryl group can be linked to the rest of the molecule through any available carbon or nitrogen atom. N atoms in the ring can be optionally oxidized forming N⁺O⁻. The heteroaryl group can be optionally substituted as disclosed above in the definitions of Cy¹, Cy² and Cy³; if substituted, the substituents can be the same or different and can be placed on any available position in the ring. Examples of heteroaryl groups include among others 1,2,4-oxadiazolyl, 1,2,4-thiadiazolyl, 1,3,4-oxadiazolyl, 1,3,4-thiadiazolyl, furyl, imidazolyl, isoxazolyl, isothiazolyl, oxazolyl, pyrazolyl, pyrrolyl, thiazolyl, thienyl, 1,2,3-triazolyl, 1,2,4-triazolyl, pyrazinyl, pyridazinyl, pyridinyl, pyrimidinyl, benzimidazolyl, benzofuranyl, benzothiazolyl, benzothiophenyl, imidazopyrazinyl, imidazopyridazinyl, imidazopyridinyl, imidazopyrimidinyl, indazolyl, indolyl, isoindolyl, isoquinolinyl, naphthiridinyl, pyrazolopyrazinyl, pyrazolopyridinyl, pyrazolopyrimidinyl, purinyl, quinazolinyl, quinolinyl and quinoxalinyl.

A heterocyclyl group means a 3- to 7-membered monocyclic carbocyclic ring or an 8- to 12-membered bicyclic carbocyclic ring which can be saturated or partially unsaturated (i.e. non-aromatic) and which contains from 1 to 4 heteratoms selected from N, S and O, and wherein said ring can be linked to the rest of the molecule through any available carbon or nitrogen atom. Additionally, one or more C or S atoms in the ring can be optionally oxidized, forming CO, SO or SO₂ groups. The heterocyclyl group can be optionally substituted as disclosed above in the definitions of Cy¹ and Cy³; if substituted, the substituents can be the same or different and can be placed on any available position in the ring. Preferably, the heterocyclyl is a 3- to 7-membered monocyclic ring. More preferably, the heterocyclyl ring has 5 or 6 ring atoms. Examples of heterocyclyl groups include, but are not limited to, aziridinyl, oxiranyl, oxetanyl, imidazolidinyl, isothiazolidinyl, isoxazolidinyl, oxazolidinyl, pyrazolidinyl, pyrrolidinyl, thiazolidinyl, dioxanyl, morpholinyl, piperazinyl, piperidinyl, pyranyl, tetrahydropyranyl, azepinyl, oxazinyl, oxazolinyl, pyrrolinyl, thiazolinyl, pyrazolinyl, imidazolinyl, isoxazolinyl, isothiazolinyl, tetrahydroisoquinolinyl, 2-oxo-pyrrolidinyl, 2-oxo-piperidinyl, 4-oxo-piperidinyl, 2-oxopiperazinyl, 2(1H)-pyridonyl, 2(1H)-pyrazinonyl, 2(1H)-pyrimidinonyl, 2(1H)-pyridazinonyl and phthalimidyl.

In the previous definition of heteroaryl, when the specified examples refer to a bicycle in general terms, all possible dispositions of the atoms are included. For example, the term pyrazolopyridinyl is to be understood as including groups such as 1H-pyrazolo[3,4-b]pyridinyl, pyrazolo[1,5-a]pyridinyl, 1H-pyrazolo[3,4-c]pyridinyl, 1H-pyrazolo[4,3-c]pyridinyl and 1H-pyrazolo[4,3-b]pyridinyl; the term imidazopyrazinyl is to be understood as including groups such as 1H-imidazo[4,5-b]pyrazinyl, imidazo[1,2-a]pyrazinyl and imidazo[1,5-a]pyrazinyl and the term pyrazolopyrimidinyl is to be understood as including groups such as 1H-pyrazolo[3,4-d]pyrimidinyl, 1H-pyrazolo[4,3-d]pyrimidinyl, pyrazolo[1,5-a]pyrimidinyl and pyrazolo[1,5-c]pyrimidinyl.

The expression “optionally substituted with one or more” means that a group can be substituted with one or more, preferably with 1, 2, 3 or 4 substituents, more preferably with 1 or 2 substituents, provided that said group has enough positions available susceptible of being substituted. When present, said substituents can be the same or different and can be placed on any available position.

In a compound of formula I, the group R₆ can be absent (n=0) or present (n=1). When R₆ is present, it can be placed on any available position on the phenyl ring.

When in a definition of a substituent two or more groups bearing the same numbering are shown (e.g. —NR₉CONR₉—, —NR₁₀R₁₀, —NR_{14′}CONR_{14′}R_{14′}, etc), this does not mean that they have to be identical. Each of them is independently selected from the list of possible meanings provided for that group, and therefore they can be the same or different.

The invention thus relates to the compounds of formula I as defined here above.

In another embodiment, the invention relates to compounds of formula I wherein A represents CR₁R₂.

In another embodiment, the invention relates to compounds of formula I wherein A represents NR₃.

In a further embodiment, the invention relates to compounds of formula I wherein m is 1.

In a further embodiment, the invention relates to compounds of formula I wherein m is 2.

In a further embodiment, the invention relates to compounds of formula I wherein A represents CR₁R₂ and m is 1.

In a further embodiment, the invention relates to compounds of formula I wherein A represents NR₃ and m is 1.

In a further embodiment, the invention relates to compounds of formula I wherein R₁ is identical to R₂.

In a further embodiment, the invention relates to compounds of formula I wherein R₁ is identical to R₂ and both represent methyl.

In a further embodiment, the invention relates to compounds of formula I wherein p is 0 or 1.

In a further embodiment, the invention relates to compounds of formula I wherein p in R₃ is 0 or 1.

In a further embodiment, the invention relates to compounds of formula I wherein p in R₈ is 0 or 1.

In a further embodiment, the invention relates to compounds of formula I wherein R₃ represents —(CH₂)ₚ—Cy¹.

In a further embodiment, the invention relates to compounds of formula I wherein R₃ represents —(CH₂)ₚ—Cy¹ and p in R₃ is 0.

In a further embodiment, the invention relates to compounds of formula I wherein R₃ represents —(CH₂)ₚ—Cy¹, p in R₃ is O and Cy¹ represents phenyl or heteroaryl, which can all be optionally substituted with one or more R₁₁.

In a further embodiment, the invention relates to compounds of formula I wherein R₃ represents —(CH₂)ₚ—Cy¹, p in R₃ is O and Cy¹ represents phenyl, which can be optionally substituted with one or more R₁₁.

In a further embodiment, the invention relates to compounds of formula I wherein R₃ represents —(CH₂)ₚ—Cy¹, p in R₃ is O and Cy¹ represents phenyl substituted with one hydroxy group and which can optionally be further substituted with one or more groups selected from R₁₁.

In a further embodiment, the invention relates to compounds of formula I wherein R₅ represents C₁₋₄ alkyl, halogen or C₁₋₄ alkoxy.

In a further embodiment, the invention relates to compounds of formula I wherein R₅ represents methyl, halogen or methoxy.

In a further embodiment, the invention relates to compounds of formula I wherein R₅ represents methyl or halogen.

In a further embodiment, the invention relates to compounds of formula I wherein n is 0.

In a further embodiment, the invention relates to compounds of formula I wherein B represents —CONH—, —NHCO— or —NHCONH—.

In a further embodiment, the invention relates to compounds of formula I wherein B represents —CONH— or —NHCO—.

In a further embodiment, the invention relates to compounds of formula I wherein B represents —CONR₉—.

In a further embodiment, the invention relates to compounds of formula I wherein R₈ represents —(CH₂)ₚ—Cy².

In a further embodiment, the invention relates to compounds of formula I wherein R₈ represents —(CH₂)ₚ—Cy² and Cy² represents C₃₋₇ cycloalkyl.

In a further embodiment, the invention relates to compounds of formula I wherein B represents —CONR₉— and R₈ represents —(CH₂)ₚ—Cy².

In a further embodiment, the invention relates to compounds of formula I wherein B represents —CONR₉—, R₈ represents —(CH₂)ₚ—Cy² and Cy² represents C₃₋₇ cycloalkyl.

In a further embodiment, the invention relates to compounds of formula I wherein B represents —CONH— and R₈ represents cyclopropyl.

Furthermore, the present invention covers all possible combinations of particular and preferred groups described hereinabove.

In a further embodiment, the invention relates to compounds according to formula I above which provide more than 50% inhibition of p38 activity at 10 μM, more preferably at 1 μM and still more preferably at 0.1 μM, in a p38 assay such as the ones described in Example 15.

In a further embodiment, the invention relates to a compound according to formula I selected from:
N-Cyclopropyl-4-methyl-3-(1-oxo-2-phenyl-2,3-dihydroisoindol-5-yl)benzamide;
N-Cyclopropylmethyl-4-methyl-3-(1-oxo-2-phenyl-2,3-dihydroisoindol-5-yl)benzamide;
3-(2-Benzyl-1-oxo-2,3-dihydroisoindol-5-yl)-N-cyclopropyl-4-methylbenzamide;
3-(2-Benzyl-1-oxo-2,3-dihydroisoindol-5-yl)-N-cyclopropylmethyl-4-methylbenzamide;
N-Cyclopropyl-3-[2-(2,2-dimethyl-3-hydroxypropyl)-1-oxo-2,3-dihydroisoindol-5-yl]-4-methylbenzamide;
N-Cyclopropyl-3-[2-(1-hydroxymethylcyclopentyl)-1-oxo-2,3-dihydroisoindol-5-yl]-4-methylbenzamide;
(1S,2S)—N-Cyclopropyl-3-[2-(2-hydroxy-1-hydroxymethyl-2-phenylethyl)-1-oxo-2,3-dihydroisoindol-5-yl]-4-methylbenzamide;
trans-N-Cyclopropyl-3-[2-(1-hydroxycyclohex-4-yl)-1-oxo-2,3-dihydroisoindol-5-yl]-4-methylbenzamide;
N-Cyclopropyl-3-[2-(2-hydroxyphenyl)-1-oxo-2,3-dihydroisoindol-5-yl]-4-methylbenzamide;
N-Cyclopropyl-3-[2-(2-hydroxy-5-sulfamoylphenyl)-1-oxo-2,3-dihydroisoindol-5-yl]-4-methylbenzamide;
N-Cyclopropyl-3-[2-(3-hydroxyphenyl)-1-oxo-2,3-dihydroisoindol-5-yl]-4-methylbenzamide;
N-Cyclopropyl-3-[2-(2-hydroxy-6-methylphenyl)-1-oxo-2,3-dihydroisoindol-5-yl]-4-methylbenzamide;
N-Cyclopropyl-4-methyl-3-(1-oxo-2-(thiazol-2-yl)-2,3-dihydroisoindol-5-yl)benzamide;
N-Cyclopropyl-3-[2-(4-hydroxyphenyl)-1-oxo-2,3-dihydroisoindol-5-yl]-4-methylbenzamide;
4-Chloro-N-cyclopropyl-3-[2-(2-hydroxyphenyl)-1-oxo-2,3-dihydroisoindol-5-yl]benzamide;
N-Cyclopropyl-3-[2-(5-chloro-2-hydroxyphenyl)-1-oxo-2,3-dihydroisoindol-5-yl]-4-methylbenzamide;
N-Cyclopropyl-3-[2-(4-chloro-2-hydroxyphenyl)-1-oxo-2,3-dihydroisoindol-5-yl]-4-methylbenzamide;
N-Cyclopropyl-3-(2-(2-hydroxyphenyl)-1-oxo-2,3-dihydroisoindol-5-yl)-4-methoxybenzamide;
N-Cyclopropyl-3-(2,2-dimethyl-1-oxoindan-5-yl)-4-methylbenzamide;
N-Cyclopropylmethyl-3-(2,2-dimethyl-1-oxoindan-5-yl)-4-methylbenzamide;
N-Butyl-3-(2,2-dimethyl-1-oxoindan-5-yl)-4-methylbenzamide;
3-(2,2-Dimethyl-1-oxoindan-5-yl)-4-methyl-N-phenylbenzamide;
3-(2,2-Dimethyl-1-oxoindan-5-yl)-4-methyl-N-(pyridin-4-yl)benzamide;
3-(2,2-Dimethyl-1-oxoindan-5-yl)-N-isopropyl-4-methylbenzamide;
3-(2,2-Dimethyl-1-oxoindan-5-yl)-4-methyl-N-(thiazol-2-yl)benzamide;
3-(2,2-Dimethyl-1-oxoindan-5-yl)-4-methyl-N-[3-(morpholin-4-yl)phenyl]benzamide;
3-(2,2-Dimethyl-1-oxoindan-5-yl)-4-methyl-N-[3-(pyridin-2-yl)phenyl]benzamide;
N-Benzyl-3-(2,2-dimethyl-1-oxoindan-5-yl)-4-methylbenzamide;
N-Cyclopropyl-3-(2-ethyl-1-oxo-1,2,3,4-tetrahydroisoquinolin-6-yl)-4-methylbenzamide;
3-(2-Benzyl-1-oxo-1,2,3,4-tetrahydroisoquinolin-6-yl)-N-cyclopropyl-4-methylbenzamide;
3-(2-Benzyl-1-oxo-1,2,3,4-tetrahydroisoquinolin-6-yl)-N-cyclopropylmethyl-4-methylbenzamide;
3-[2-(2-Chlorophenyl)-1-oxo-1,2,3,4-tetrahydroisoquinolin-6-yl]-N-cyclopropyl-4-methylbenzamide;
N-Cyclopropyl-3-(2,2-dimethyl-1-oxo-1,2,3,4-tetrahydronaphthalen-6-yl)-4-methylbenzamide;
N-Cyclopropylmethyl-3-(2,2-dimethyl-1-oxo-1,2,3,4-tetrahydronaphthalen-6-yl)-4-methylbenzamide;
N-Cyclopropyl-3-[2-(2-hydroxyethyl)-1-oxo-2,3-dihydroisoindol-5-yl]-4-methylbenzamide;
N-Cyclopropyl-4-methyl-3-(1-oxo-2-(pyridin-4-ylmethyl)-2,3-dihydroisoindol-5-yl)benzamide;
N-Cyclopropyl-4-methyl-3-[2-(3-nitrobenzyl)-1-oxo-2,3-dihydroisoindol-5-yl]benzamide;
3-[2-(3-Cyanophenyl)-1-oxo-2,3-dihydroisoindol-5-yl]-N-cyclopropyl-4-methylbenzamide;
N-Cyclopropyl-4-methyl-3-[2-(3-(morpholin-4-yl)phenyl)-1-oxo-2,3-dihydroisoindol-5-yl]benzamide;
3-(2-(Biphenyl-3-yl)-1-oxo-2,3-dihydroisoindol-5-yl)-N-cyclopropyl-4-methylbenzamide;
N-Cyclopropyl-3-[2-(3-hydroxypropyl)-1-oxo-2,3-dihydroisoindol-5-yl]-4-methylbenzamide;
N-Cyclopropyl-4-methyl-3-[2-(2-(morpholin-4-yl)ethyl)-1-oxo-2,3-dihydroisoindol-5-yl]benzamide;
N-Cyclopropyl-4-methyl-3-[1-oxo-2-(2-pyridin-3-ylethyl)-2,3-dihydroisoindol-5-yl]benzamide;
N-Cyclopropyl-3-[2-(indazol-6-yl)-1-oxo-2,3-dihydroisoindol-5-yl]-4-methylbenzamide;
N-Cyclopropyl-3-[2-(indol-5-yl)-1-oxo-2,3-dihydroisoindol-5-yl]-4-methylbenzamide;
3-[2-(1-Acetylpiperidin-4-yl)-1-oxo-2,3-dihydroisoindol-5-yl]-N-cyclopropyl-4-methylbenzamide;
N-Cyclopropyl-3-[2-(6-methoxypyridin-3-yl)-1-oxo-2,3-dihydroisoindol-5-yl]-4-methylbenzamide;
N-Cyclopropyl-3-[2-ethyl-1-oxo-2,3-dihydroisoindol-5-yl]-4-methylbenzamide;
N-Cyclopropyl-3-[2-(2-methoxyphenyl)-1-oxo-2,3-dihydroisoindol-5-yl]-4-methylbenzamide;
N-Cyclopropyl-5-fluoro-3-[2-(2-hydroxyphenyl)-1-oxo-2,3-dihydroisoindol-5-yl]-4-methylbenzamide;
N-Cyclopropyl-5-fluoro-3-[2-(2,2-dimethyl-3-hydroxypropyl)-1-oxo-2,3-dihydroisoindol-5-yl]-4-methylbenzamide;
2-Cyclopropyl-N-[4-methyl-3-(1-oxo-2-phenyl-2,3-dihydroisoindol-5-yl)phenyl]acetamide;
N-[4-Methyl-3-(1-oxo-2-phenyl-2,3-dihydroisoindol-5-yl)phenyl]furan-3-carboxamide;
N-[3-(2,2-Dimethyl-1-oxoindan-5-yl)-4-methylphenyl]cyclopropylcarboxamide;
2-Cyclopropyl-N-[3-(2,2-dimethyl-1-oxoindan-5-yl)-4-methylphenyl]acetamide;
2-Chloro-N-[3-(2,2-dimethyl-1-oxoindan-5-yl)-4-methylphenyl]isonicotinamide;
N-[3-(2,2-Dimethyl-1-oxoindan-5-yl)-4-methylphenyl]thiophene-3-carboxamide;
N-[3-(2,2-Dimethyl-1-oxoindan-5-yl)-4-methylphenyl]furan-3-carboxamide;
N-[3-(2,2-Dimethyl-1-oxoindan-5-yl)-4-methylphenyl]-2-(pyrrolidin-1-yl)isonicotinamide;
N-[3-(2,2-Dimethyl-1-oxoindan-5-yl)-4-methylphenyl]-2-(morpholin-4-yl)isonicotinamide;
1-Benzyl-3-[3-(2,2-dimethyl-1-oxoindan-5-yl)-4-methylphenyl]urea;
1-[3-(2,2-Dimethyl-1-oxoindan-5-yl)-4-methylphenyl]-3-isopropylurea;
3-[2-(3-Aminobenzyl)-1-oxo-2,3-dihydroisoindol-5-yl]-N-cyclopropyl-4-methylbenzamide;
N-Cyclopropyl-3-[2-(3-methanesulfonylaminobenzyl)-1-oxo-2,3-dihydroisoindol-5-yl]-4-methylbenzamide; and
3-(2-Benzyl-1-oxo-1,2,3,4-tetrahydroisoquinolin-6-yl)-N-cyclopropylbenzamide.

The compounds of the present invention may contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when they are used for therapeutic purposes. The term pharmaceutically acceptable salt represents those salts which are, according to medical judgement, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in the conventional manner. The salts of the compounds of formula I can be converted into other salts of the compounds of formula I by ion exchange using ionic exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof and a solvent. Examples of solvents include pharmaceutically acceptable solvents such as water, ethanol and the like. A complex with water is known as a hydrate. Solvates of compounds of the invention (or salts thereof, including hydrates, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of general formula I. Optically pure isomers can also be individually obtained using enantiospecific synthesis. The present invention covers all individual isomers as well as mixtures thereof (for example racemic mixtures or mixtures of diastereomers), whether obtained by synthesis or by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups by conventional protective groups. Both the nature of these protective groups and the procedures for their introduction or removal are well known in the art (see for example Greene T. W. and Wuts P. G. M, “Protective Groups in Organic Synthesis”, John Wiley & Sons, 3^{rd} edition, 1999). As an example, as protective groups of an amino function tert-butoxycarbonyl (Boc) or benzyl (Bn) groups can be used. The carboxyl groups can be protected for example in the form of C₁₋₄ alkyl esters or arylalkyl esters, such as benzyl, while the hydroxyl groups can be protected for example with tetrahydropyranyl (THP) or benzyl (Bn) groups. Whenever a protective group is present, a later deprotection step will be required, which can be performed under standard conditions in organic synthesis, such as those described in the above-mentioned reference.

Unless otherwise stated, in the methods described below the meanings of the differents substituents are the meanings described above with regard to a compound of general formula I.

Most of the compounds of formula I can be obtained by reacting a compound of formula II with a compound of formula III, as shown in the following scheme:

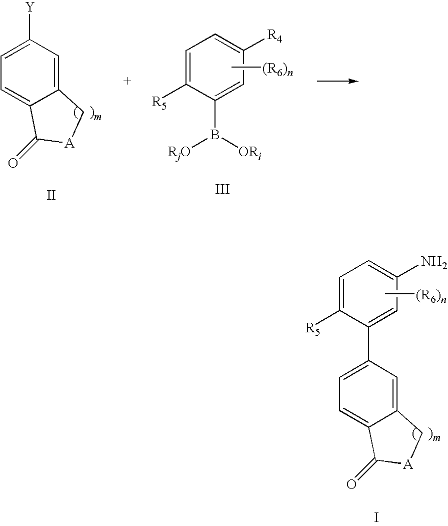

wherein A, R₄, R₅, R₆, m and n have the meaning described above in connection with a compound of general formula I, Y represents halogen, preferably bromo, or trifluoromethanesulfonate, and each Rᵢ and Rⱼ represent H or C₁₋₄alkyl or they can be linked together to form together with the B and O atoms a five or six-membered ring which can be optionally substituted by one or more methyl groups. This reaction is carried out in the presence of a base, such as K₂CO₃, Na₂CO₃, CsF or K₃PO₄, and a palladium catalyst, such as Pd(PPh₃)₄, in a solvent such as dimethoxyethane, dioxane, diglyme or dimethylformamide, optionally in the presence of water, and heating, preferably at reflux.

Alternatively, a compound of formula I wherein R₄═—CONR₉R₈ (Ia) can be obtained from a compound of formula IV and an amine of formula V, as shown in the following scheme:

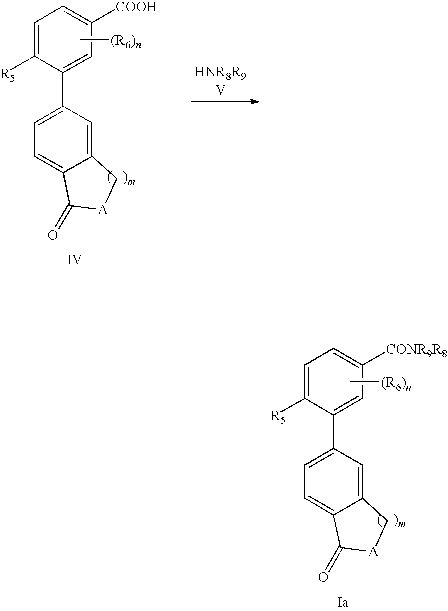

wherein A, R₅, R₆, R₈, R₉, m and n have the meaning described above. This reaction is carried out in the presence of an activating agent such as (benzotriazol-1-yloxy)tripyrrolidinophosphonium hexafluorophosphate, N-(3-dimethylaminopropyl)-N′-ethylcarbodiimide hydrochloride or N,N-dicyclohexylcarbodiimide and 1-hydroxybenzotriazol, and in the presence of a base such as N,N-diisopropylethylamine or N-methylmorpholine and in a suitable solvent such as dimethylformamide. Alternatively, the reaction can be carried out by conversion of the carboxylic acid of formula IV into an acyl chloride, by using standard conditions in organic synthesis, followed by conversion of the latter into the amide of formula Ia by reaction with an amine of formula V in the presence of a base such as triethylamine, in a suitable solvent such as for example dichloromethane, and cooling, preferably at 0° C.

Compounds of formula IV can be obtained by reacting a compound of formula II with a compound of formula IIIa, as shown in the following scheme:

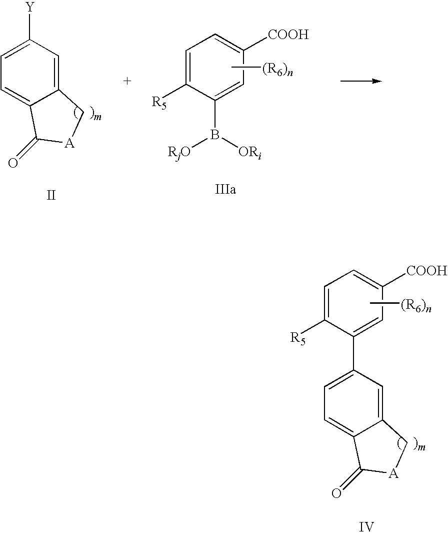

wherein A, R₅, R₆, m, n, Y, Rᵢ and Rⱼ have the meaning described above. This reaction is carried out under the same conditions described above for the preparation of compounds I from compounds II and III.

The compounds of formula I wherein R₄═—NHCOR₈ (Ib) can be obtained from a compound of formula VI and an acid of formula VII, as shown in the following scheme:

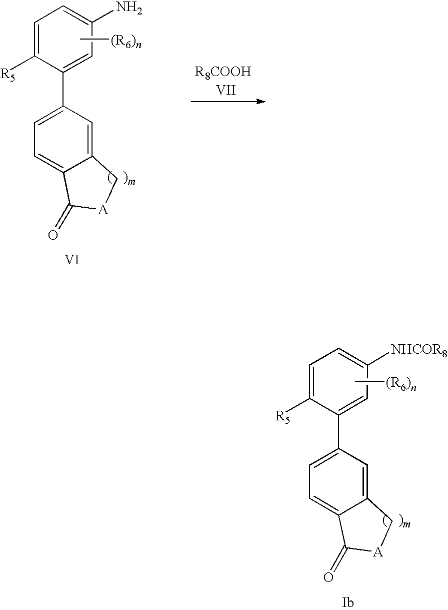

wherein A, R₅, R₆, R₈, m and n have the meaning described above. This reaction is carried out under the same conditions described above for the preparation of compounds Ia from compounds IV and V.

The compounds of formula I wherein R₄═—NR₉COR₈ and R₉═C₁₋₄ alkyl may be obtained from the corresponding compound of formula Ib by alkylation under basic conditions, following standard procedures.

Compounds of formula VI can be obtained from compounds of formula IV, as shown in the following scheme:

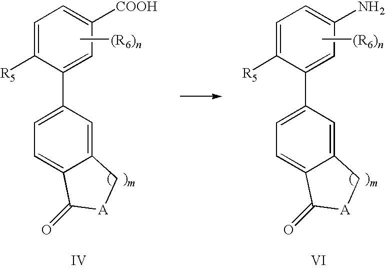

wherein A, R₅, R₆, m and n have the meaning described above. This reaction can be carried out under standard Curtius conditions, for example by treatment with diphenylphosphorylazide, in the presence of a base, such as for example triethylamine, in a suitable solvent, such as dimethylformamide and at a suitable temperature, preferably 100° C., followed by aqueous treatment.

The compounds of formula I wherein R₄═—NHCONR₉R₈ (Ic) can be obtained from a compound of formula VI, as shown in the following scheme:

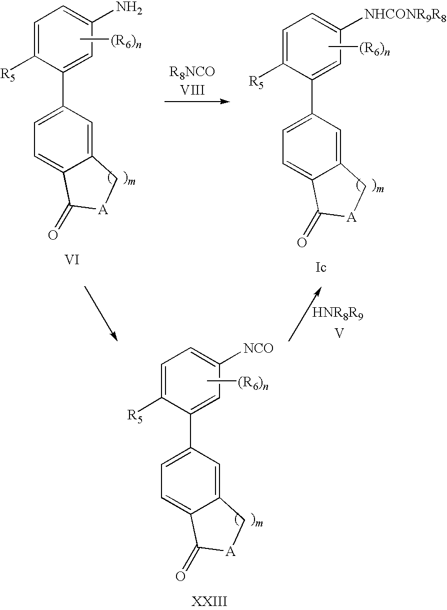

wherein A, R₅, R₆, R₈, R₉, m and n have the meaning described above. The compounds of formula Ic wherein R₉═H can be obtained by reaction of compound VI with an isocyanate of formula VIII. This reaction is carried out in a suitable solvent, such as dimethylformamide, and at a suitable temperature comprised between room temperature and the temperature of the boiling point of the solvent. Alternatively, a compound of formula Ic can be obtained from a compound of formula VI by a two step sequence which involves converting the amine into the corresponding isocyanate (XXIII) with triphosgene, in the presence of a base such as N,N-diisopropylethylamine, triethylamine or N-methylmorpholine, in a suitable solvent such as acetonitrile or a halogenated hydrocarbon such as chloroform or dichloromethane; and then reacting the resulting isocyanate XXIII with an amine of formula V in a suitable solvent, such as the solvent used in the first step.

Compounds of formula II wherein A=CR₁R₂ (IIa: A=CR₁R₂, m=1; IIb: A=CR₁R₂, m=2) and Y represents halogen can be obtained by reacting a compound of formula IX with an alkylating agent of formula X, as shown in the following scheme:

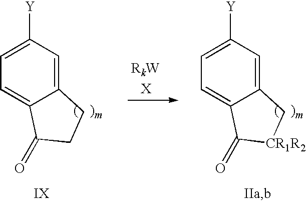

wherein R₁, R₂ and m have the meaning described above, Y represents halogen, preferably bromo, Rₖ represents R₁ or R₂ and W represents halogen or alkylsulfonate, preferably iodo. This reaction can be carried out in the presence of a base such as sodium hydride, in a suitable solvent such as toluene, tetrahydrofuran or dimethylformamide, and at a temperature comprised between room temperature and the temperature of the boiling point of the solvent. When R₁≠R₂, this reaction is carried out in a two-step sequence that involves alkylating a compound of formula IX with an alkylating agent R₁W to give a mono-alkylated intermediate and then reacting this intermediate with a second alkylating agent R₂W to yield the compound of formula IIa,b.

Compounds of formula II wherein A=NR₃ and m=1 (IIc) can be obtained by reacting a compound of formula XIa with an amine of formula XII, as shown in the following scheme:

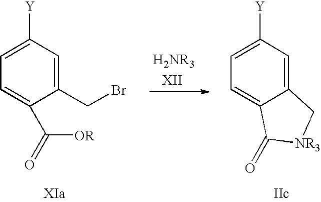

wherein R₃ has the meaning described above, R represents C₁₋₄ alkyl and Y represents halogen, preferably bromo. This reaction can be carried out in a suitable solvent such as methanol, ethanol or dimethylformamide, optionally in the presence of a base such as a tertiary amine (like triethylamine or N,N-diisopropylethylamine), sodium carbonate or potassium carbonate, and at a temperature comprised between room temperature and the temperature of the boiling point of the solvent. Alternatively, this reaction can be carried out in a two-step sequence that involves bromo displacement from a compound of formula XIa by the amine XII in a suitable solvent such as methanol, ethanol or dimethylformamide, to yield an intermediate aminoester, and final cyclization to the compound of formula IIc by heating in acetic acid or polyphosphoric acid.

Compounds of formula II wherein Y represents trifluoromethanesulfonate can be obtained starting from a compound of formula XIII, as shown in the following scheme:

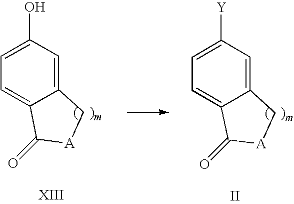

wherein A and m have the meaning described above and Y represents trifluoromethanesulfonate. This reaction can be carried out in the presence of a suitable sulfonylating agent such as trifluoromethanesulfonic anhydride or trifluoromethanesulfonyl chloride, in a suitable solvent such as pyridine or dichloromethane, in the presence of a base such as pyridine or triethylamine, and at a suitable temperature comprised between 0° C. and room temperature.

Compounds of formula XIII can be obtained starting from a compound of formula XIV, as shown in the following scheme:

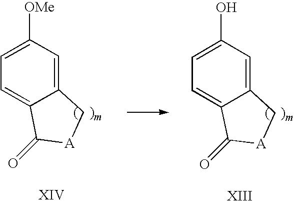

wherein A and m have the meaning described above. This reaction can be carried out in the presence of a strong acid, such as 48% HBr, and at a suitable temperature comprised between room temperature and the temperature of the boiling point of the solvent, or in the presence of a Lewis acid such as boron tribromide, in a suitable solvent such as dichloromethane, and at a temperature comprised preferably between −78° C. and room temperature.

Compounds of formula XIV wherein A=CR₁R₂ (XIVa: A=CR₁R₂, m=1; XIVb: A=CR₁R₂, m=2) can be obtained by reaction of compounds of formula XV under the same conditions previously described for the conversion of a compound of formula IX into a compound of formula IIa,b, as shown in the following scheme:

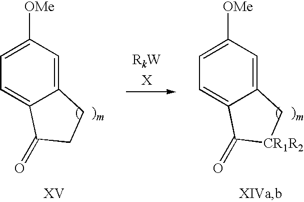

wherein R₁, R₂ and m have the meaning described above.

Compounds of formula XIV wherein A=NR₃ and m=1 (XIVc) can be obtained by reacting a compound of formula XIb with an amine of formula XII, as shown in the following scheme:

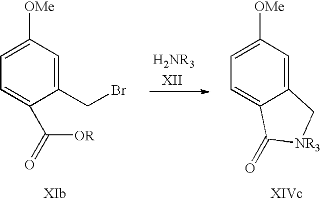

wherein R and R₃ have the meaning described above. This reaction can be carried out under the same reaction conditions described above for the preparation of compounds IIc from XIa.

Compounds of formula XIa,b can be obtained starting from a compound of formula XVI, as shown in the following scheme:

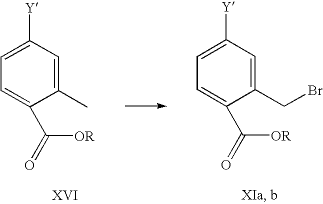

wherein R has the meaning described above and Y′ represents halogen, preferably bromo, or methoxy. This reaction can be carried out in the presence of a suitable halogenating agent, such as N-bromosuccinimide, optionally in the presence of a radical initiator such as 2,2′-azobis(2-methylbutyronitrile) or benzoyl peroxide, in a suitable solvent such as CCl₄, CHCl₃, acetonitrile or chlorobenzene, and at a suitable temperature comprised between room temperature and the temperature of the boiling point of the solvent, optionally irradiating the mixture.

Compounds of formula XVI can be obtained by reacting a carboxylic acid of formula XVII with an alcohol of formula XVIII, as shown in the following scheme:

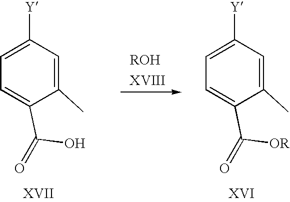

wherein R has the meaning described above and Y′ represents halogen, preferably bromo, or methoxy. This reaction can be carried out in the presence of an inorganic acid such as concentrated sulfuric acid, using the alcohol of formula XVIII as the solvent, and at a suitable temperature comprised between room temperature and the temperature of the boiling point of the solvent. Alternatively, a compound of formula XVII can be converted into the corresponding acyl chloride by using standard conditions and then the latter can be converted into the corresponding ester of formula XVI by reaction with an alcohol of formula XVIII, in the presence of a base such as triethylamine, in a suitable solvent such as for example dichloromethane, and at a suitable temperature comprised between 0° C. and room temperature.

Compounds of formula XIV wherein A=NR₃ (XIVC: m=1; XIVd: m=2) can be obtained starting from a compound of formula XIX, as shown in the following scheme:

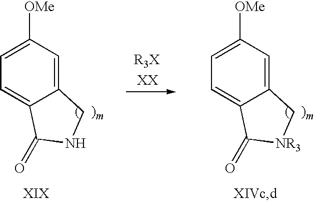

wherein R₃ and m have the meaning described above. When R₃ is an alkyl-type group, this reaction can be carried out by treatment with an alkylating agent such as a halide or alkylsulfonate of formula XX, preferably an alkyl iodide, in the presence of a base such as sodium hydride, in a suitable solvent such as toluene, tetrahydrofuran or dimethylformamide, and at a temperature comprised between room temperature and the temperature of the boiling point of the solvent. When R₃ is a phenyl or heteroaryl group, this reaction can be carried out by reaction with an halide of formula XX, preferably a bromide, in the presence of a base, such as K₂CO₃, Na₂CO₃ or K₃PO₄, and a copper catalyst, such as copper(I) iodide, in a solvent such as N-methylpyrrolidone and heating, preferably at reflux.

Alternatively, compounds of formula II wherein A=NR₃ (IIc: m=1; IId: m=2) can be obtained in an analogous manner starting from a compound of formula XXI, as shown in the following scheme:

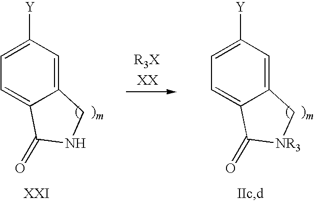

wherein R₃ and m have the meaning described above and Y represents halogen, preferably bromo. This reaction is carried out under the same reaction conditions described above for the preparation of compounds XIVc,d from XIX.

Compounds of formula III are either commercially available or can be obtained starting from a compound of formula XXII, as shown in the following scheme:

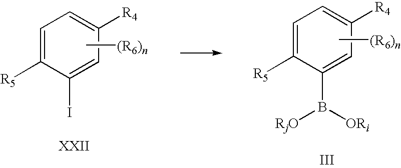

wherein A, R₄, R₅, R₆, n, Rᵢ and R^{j} have the meaning described above. This reaction is carried out in the presence of a boron reagent such as bis(pinacolato)diboron, a palladium catalyst such as [1,1′-bis(diphenylphosphino)ferrocene]dichloro-palladium (II) and a base such as potassium acetate, in a suitable solvent such as dimethylformamide, dimethoxyethane or dioxane, and at a suitable temperature, comprised between room temperature and the temperature of the boiling point of the solvent, preferably heating; or alternatively in the presence of a trialkylborate and a strong base, such as butyllithium, in a suitable solvent such as tetrahydrofuran, and at a suitable temperature, preferably cooling at −78° C., optionally followed by hydrolysis of the boronic ester to yield the corresponding boronic acid.

Compounds of formulae V, VII, VIII, IX, X, XII, XV, XVII, XVIII, XIX, XX, XXI and XXII are commercially available or can be prepared by methods widely described in the literature, and can be conveniently protected.

Furthermore, some compounds of the present invention can also be obtained from other compounds of formula I by appropriate conversion reactions of functional groups in one or several steps, using well-known reactions in organic chemistry under the reported standard experimental conditions.

Such interconversions can be carried out upon groups R₃ or R₄ and include, for example:

the conversion of a nitro group into an amine by reaction with a reducing agent such as hydrogen in the presence of a Pd catalyst such as Pd on activated carbon or a metal reducing agent such as tin (II) chloride or iron, in a suitable solvent such as methanol, ethanol or acetic acid;

the conversion of an amine into a sulfonamide by reaction with a sulfonyl halide, such as sulfonyl chloride, optionally in the presence of catalytic amounts of a base such as 4-dimethylaminopyridine, in a suitable solvent such as dioxane, chloroform, dichloromethane or pyridine, optionally in the presence of a base such as triethylamine or pyridine;

the conversion of an amine into an amide, carbamate or urea under standard conditions, for example following the methods disclosed above;

the conversion of an aromatic halide into an aromatic amine by reaction with an amine, optionally in the presence of a suitable solvent, and preferably heating;

the alkylation of an amide by treatment with an alkylating agent under basic conditions.

Some of these interconversion reactions are explained in greater detail in the examples.

As it will be obvious to those skilled in the art, these interconversion reactions can be carried out upon the compounds of formula I as well as upon any suitable synthesis intermediate thereof.

As mentioned previously, the compounds of the present invention act as p38 kinase inhibitors, inducing the reduction of proinflammatory cytokines. Therefore, the compounds of the invention are expected to be useful to treat or prevent diseases in which p38 plays a role in mammals, including human beings. This includes diseases caused by overproduction of cytokines such as TNF-α, IL-1, IL-6 or IL-8. These diseases include, but are not limited to, immune, autoimmune and inflammatory diseases, cardiovascular diseases, infectious diseases, bone resorption disorders, neurodegenerative diseases, proliferative diseases and processes associated with cyclooxygenase-2 induction. Preferred diseases to be treated or prevented with the compounds of the invention are immune, autoimmune and inflammatory diseases.

As an example, immune, autoimmune and inflammatory diseases that can be treated or prevented with the compounds of the present invention include rheumatic diseases (e.g. rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis), glomerulonephritis (with or without nephrotic syndrome), autoimmune hematologic disorders (e.g. hemolytic anemia, aplasic anemia, idiopathic thrombocytopenia and neutropenia), autoimmune gastritis and autoimmune inflammatory bowel diseases (e.g. ulcerative colitis and Crohn's disease), host versus graft disease, allograft rejection, chronic thyroiditis, Graves' disease, schleroderma, diabetes (type I and type II), active hepatitis (acute and chronic), primary biliary cirrhosis, myasthenia gravis, multiple sclerosis, systemic lupus erythematosus, psoriasis, atopic dermatitis, contact dermatitis, eczema, skin sunburns, chronic renal insufficiency, Stevens-Johnson syndrome, idiopathic sprue, sarcoidosis, Guillain-Barré syndrome, uveitis, conjunctivitis, keratoconjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease (e.g. chronic obstructive pulmonary disease) and other inflammatory or obstructive diseases of the airways.

Cardiovascular diseases that can be treated or prevented include, among others, myocardial infarction, cardiac hypertrophy, cardiac insufficiency, ischaemia-reperfusion disorders, thrombosis, thrombin-induced platelet aggregation, acute coronary syndromes, atherosclerosis and cerebrovascular accidents.

Infectious diseases that can be treated or prevented include, among others, sepsis, septic shock, endotoxic shock, sepsis by Gram-negative bacteria, shigellosis, meningitis, cerebral malaria, pneumonia, tuberculosis, viral myocarditis, viral hepatitis (hepatitis A, hepatitis B and hepatitis C), HIV infection, retinitis caused by cytomegalovirus, influenza, herpes, treatment of infections associated with severe burns, myalgias caused by infections, cachexia secondary to infections, and veterinary viral infections such as lentivirus, caprine arthritic virus, visna-maedi virus, feline immunodeficiency virus, bovine immunodeficiency virus or canine immunodeficiency virus.

Bone resorption disorders that can be treated or prevented include osteoporosis, osteoarthritis, traumatic arthritis and gouty arthritis, as well as bone disorders related with multiple myeloma, bone fracture and bone grafting and, in general, all these processes wherein it is necessary to induce osteoblastic activity and increase bone mass.

Neurodegenerative diseases that can be treated or prevented include Alzheimer's disease, Parkinson's disease, cerebral ischaemia and traumatic neurodegenerative disease, among others.

Proliferative diseases that can be treated or prevented include endometriosis, solid tumors, acute and chronic myeloid leukemia, Kaposi sarcoma, multiple myeloma, metastatic melanoma and angiogenic disorders such as ocular neovascularisation and infantile haemangioma.

p38 kinase inhibitors also inhibit the expression of proinflammatory proteins such as cyclooxygenase-2 (COX-2), the enzyme responsible for prostaglandin production. Therefore, the compounds of the present invention can also be used to treat or prevent diseases mediated by COX-2 and especially to treat processes with edema, fever and neuromuscular pain such as cephalea, pain caused by cancer, tooth pain, arthritic pain, hyperalgesia and allodynia.

In vitro and in vivo assays to determine the ability of a compound to inhibit p38 activity are well known in the art. For example, a compound to be tested can be contacted with the purified p38 enzyme to determine whether inhibition of p38 activity occurs. Alternatively, cell-based assays can be used to measure the ability of a compound to inhibit the production of cytokines such as TNFalpha, e.g. in stimulated peripheral blood mononuclear cells (PBMCs) or other cell types. Detailed disclosure of assays that can be used to test the biological activity of the compounds of the invention as p38 inhibitors can be found below (see Example 15).

For selecting active compounds, testing at 10 μM must result in an activity of more than 50% inhibition in at least one of the tests provided in Example 15. More preferably, compounds should exhibit more than 50% inhibition at 1 μM, and still more preferably, they should exhibit more than 50% inhibition at 0.1 μM.

The present invention also relates to a pharmaceutical composition which comprises a compound of the present invention (or a pharmaceutically acceptable salt or solvate thereof and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the addition of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as purified water, ethanol, sorbitol, glycerol, polyethylene glycols (macrogols) and propylene glycol. Said compositions can also contain coadjuvants such as wetting, suspending, sweetening, flavouring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions which will be dissolved in water or any other sterile injectable medium immediately before use. It is also possible to start from sterile materials and keep them under these conditions throughout all the manufacturing process.

For the rectal administration, the active compound can be preferably formulated as a suppository on an oily base, such as for example vegetable oils or solid semisynthetic glycerides, or on a hydrophilic base such as polyethylene glycols (macrogol).

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as a single or divided doses.

The invention is illustrated by the following examples.

### EXAMPLES

The following abbreviations have been used in the examples:
ACN: acetonitrile
DMF: dimethylformamide
DMSO: dimethylsulfoxide
EDC.HCl: N-(3-dimethylaminopropyl)-N′-ethylcarbodiimide hydrochloride
EtOAc: ethyl acetate
EtOH: ethanol
HOAc: acetic acid
HOBT: 1-hydroxybenzotriazole hydrate
MeOH: methanol
PyBOP: (Benzotriazol-1-yloxy)tripyrrolidinophosphonium hexafluorophosphate
TEA: triethylamine
THF: tetrahydrofuran
t_{R}: retention time
LC-MS: liquid chromatography-mass spectrometry

LC-MS spectra have been performed using the following chromatographic method:

Method 1: Column Tracer Excel 120, ODSB 5 μm (10 mm×0.21 mm), temperature: 30° C., flow: 0.35 mL/min, eluent: A=ACN, B=0.1% HCOOH, gradient: 0 min 10% A-10 min 90% A-15 min 90% A
Method 2: Column X-Terra MS C18 5 μm (100 mm×2.1 mm), temperature: 30° C., flow: 0.35 mL/min, eluent: A=ACN, B=10 mM Ammonium bicarbonate, gradient: 0 min 10% A-10 min 90% A-15 min 90% A.
Method 3: Column X-Terra MS C18 5 μm (150 mm×2.1 mm), temperature: 30° C., flow: 0.35 mL/min, eluent: A=ACN, B=0.1% HCOOH, gradient: 0 min 10% A-10 min 90% A-15 min 90% A.

The MS spectra have been obtained with positive electrospray ionization mode over a scan range from 100 to 800 amu.

### Reference Example 1

### Methyl 4-bromo-2-methylbenzoate

To a solution of 4-bromo-2-methylbenzoic acid (6.17 g, 0.29 mol) in MeOH (170 mL), H₂SO₄ 95% (3 mL) was added. It was heated to reflux overnight and allowed to cool to room temperature. The solvent was evaporated and EtOAc was added. The organic phase was washed with saturated NaHCO₃, aq Na₂CO₃ and water. The combined organic phases were dried over Na₂SO₄ and the solvent was evaporated, to afford 6.43 g of the title compound as an oil (yield: 98%).

¹H NMR (300 MHz, CDCl₃) δ (TMS): 2.58 (s, 3H), 3.89 (s, 3H), 7.36 (d, J=1.8 Hz, 1H), 7.41 (dd, J=8.1 Hz, J′=1.8 Hz, 1H), 7.78 (d, J=8.1 Hz, 1H).

### Reference Example 2

### Methyl 4-bromo-2-(bromomethyl)benzoate

To a solution of methyl 4-bromo-2-methylbenzoate (9.60 g, 0.42 mol, obtained in reference example 1) in CCl₄ (150 mL), N-bromosuccinimide (7.46 g, 0.42 mol) and benzoyl peroxide (0.19 g, 0.79 mmol) were added. The reaction mixture was stirred 4 h at room temperature while irradiated with a 250 Watt lamp and it was then filtered to remove the precipitated solids. The filtrate was washed with 1N NaOH and water and it was dried over Na₂SO₄. The solvent was evaporated to afford 11.87 g of the desired compound as an oil that solidified on standing (yield: 92%, uncorrected).

¹H NMR (300 MHz, CDCl₃) δ (TMS): 3.94 (s, 3H), 4.90 (s, 2H), 7.51 (dd, J=8.4 Hz, J′=2.1 Hz, 1H), 7.63 (d, J=1.8 Hz, 1H), 7.84 (d, J=8.4 Hz, 1H).

### Reference Example 3

### 5-Bromo-2-phenyl-2,3-dihydroisoindol-1-one

To a solution of methyl 4-bromo-2-(bromomethyl)benzoate (4.9 mmol, obtained in reference example 2) in MeOH (40 mL), aniline (0.93 g, 5.1 mmol) and TEA (1.05 mL, 7.6 mmol) were added. The mixture was heated to reflux for 24 h and then allowed to cool to room temperature. The solvent was evaporated and the crude product obtained was purified by chromatography on silica gel using hexane-EtOAc mixtures of increasing polarity as eluent, to afford 1.07 g of the desired compound, impurified with starting aniline. The product was dissolved in CHCl₃ and the organic phase was washed with 1N HCl, dried over Na₂SO₄ and the solvent evaporated to afford 0.98 g of the title compound (yield: 67%).

¹H NMR (300 MHz, CDCl₃) δ (TMS): 4.85 (s, 2H), 7.18 (m, 1H), 7.46 (m, 2H), 7.64-7.86 (complex signal, 5H)

### Reference Examples 3A-3P, 3AA-3AC

Following a similar procedure to that described in reference example 3, but starting from the appropriate amine in each case, the compounds in the following table were obtained:

| | | | LC-MS | | |
|---|---|---|---|---|---|
| Reference | | | | t_{R} | m/z |
| example | Compound name | Starting amine | Method | (min) | [M + H]⁺ |
| 3A | 2-Benzyl-5-bromo-2,3- | Benzylamine | 1 | 8.69 | 302.0/ |
| | dihydroisoindol-1-one | | | | 304.0 |
| 3B | 5-Bromo-2-(pyridin-4- | 4- | 1 | 4.01 | 303.0/ |
| | ylmethyl)-2,3-dihydroisoindol- | Aminomethylpyridine | | | 305.0 |
| | 1-one | | | | |
| 3C | 5-Bromo-2-(3-nitrobenzyl)- | 3-Nitrobenzylamine | 1 | 8.32 | 347.0/ |
| | 2,3-dihydroisoindol-1-one | hydrochloride | | | 349.0 |
| 3D | 5-Bromo-2-(3-cyanophenyl)- | 3-Aminobenzonitrile | 1 | — | NMR |
| | 2,3-dihydroisoindol-1-one | | | | (see |
| | | | | | below) |
| 3E | 5-Bromo-2-(3-(morpholin-4- | Reference example | 1 | 8.72 | 373.1/ |
| | yl)phenyl)-2,3-dihydroisoindol- | 30 | | | 375.1 |
| | 1-one | | | | |
| 3F | 2-(Biphenyl-3-yl)-5-bromo-2,3- | 3-Aminobiphenyl | 1 | 10.79 | 364.0/ |
| | dihydroisoindol-1-one | | | | 366.1 |
| 3G | 5-Bromo-2-(3-hydroxypropyl)- | 3-Amino-1-propanol | 1 | 5.23 | 270.0/ |
| | 2,3-dihydroisoindol-1-one | | | | 272.0 |
| 3H | 5-Bromo-2-(2-hydroxyethyl)- | Ethanolamine | 1 | 4.89 | 256.0/ |
| | 2,3-dihydroisoindol-1-one | | | | 258.0 |
| 3I | 5-Bromo-2-(2-morpholin-4- | 2- | 1 | 3.70 | 325.1/ |
| | ylethyl)-2,3-dihydroisoindol-1- | Morpholinoethylamine | | | 327.1 |
| | one | | | | |
| 3J | 5-Bromo-2-(2-pyridin-3- | 2-(Pyridin-3- | 1 | 4.07 | 317.1/ |
| | ylethyl)-2,3-dihydroisoindol-1- | yl)ethylamine | | | 319.1 |
| | one | | | | |
| 3K | 5-Bromo-2-(indol-5-yl)-2,3- | 5-Aminoindole | 1 | 8.63 | 327.1/ |
| | dihydroisoindol-1-one | | | | 329.1 |
| 3L | 5-Bromo-2-(1- | 1-Amino-1- | 1 | 7.14 | 310.0/ |
| | hydroxymethylcyclopentyl)- | cyclopentane- | | | 312.0 |
| | 2,3-dihydroisoindol-1-one | methanol | | | |
| 3M | 5-Bromo-2-(2,2-dimethyl-3- | 3-Amino-2,2- | 1 | 7.41 | 298.1/ |
| | hydroxypropyl)-2,3- | dimethylpropanol | | | 300.1 |
| | dihydroisoindol-1-one | | | | |
| 3N | 5-Bromo-2-(2-hydroxyphenyl)- | 2-Aminophenol | 1 | 7.55 | 304.0/ |
| | 2,3-dihydroisoindol-1-one | | | | 306.0 |
| 3O | 5-Bromo-2-(3-hydroxyphenyl)- | 3-Aminophenol | 1 | 7.44 | 302.0/ |
| | 2,3-dihydroisoindol-1-one | | | | 304.0 |
| | | | | | [M − H]⁻ |
| 3P | 5-Bromo-2-(6-methoxypyridin- | 5-Amino-2- | 1 | 8.09 | 319.0/ |
| | 3-yl)-2,3-dihydroisoindol-1- | methoxypyridine | | | 321.0 |
| | one | | | | |
| 3AA | 5-Bromo-2-(2- | 2- | 2 | 7.68 | 318.1/ |
| | methoxyphenyl)-2,3- | methoxyphenylamine | | | 320.1 |
| | dihydroisoindol-1-one | | | | |
| 3AB | 5-Bromo-2-(5-chloro-2- | 2-amino-4- | 2 | 7.76 | 338.0/ |
| | hydroxyphenyl)-2,3- | chlorophenol | | | 340.1 |
| | dihydroisoindol-1-one | | | | |
| 3AC | 5-Bromo-2-(4-chloro-2- | 2-amino-5- | 2 | 7.62 | 338.0/ |
| | hydroxyphenyl)-2,3- | chlorophenol | | | 340.1 |
| | dihydroisoindol-1-one | | | | |

Reference example 3D: ¹H NMR (300 MHz, CDCl₃) δ (TMS): 4.86 (s, 2H), 7.45-7.60 (complex signal, 2H), 7.71 (m, 2H), 7.80 (m, 1H), 8.19 (m, 2H).

### Reference Example 3Q

### (1S,2S)-5-Bromo-2-(2-hydroxy-1-hydroxymethyl-2-phenylethyl)-2,3-dihydroisoindol-1-one

To a solution of methyl 4-bromo-2-(bromomethyl)benzoate (0.8 mmol, obtained in reference example 2) in MeOH (7 mL), (1S,2S)-2-amino-1-phenyl-1,3-propanediol (0.27 g, 1.6 mmol) was added. The mixture was heated to reflux overnight and then allowed to cool to room temperature. The solvent was evaporated and the crude product thus obtained was slurried in CHCl₃ and filtered. The solids were washed with CHCl₃ and water, and then dried under vacuum to afford 0.13 g of the title compound (yield: 45%).

LC-MS (method 1): t_{R}=6.54 min; m/z=362.0/364.0 [M+H]⁺.

### Reference Examples 3R-3Y

Following a similar procedure to that described in reference example 3Q, but starting from the appropriate amine in each case, the compounds in the following table were obtained:

| | | | LC-MS | | |
|---|---|---|---|---|---|
| Reference | | | | t_{R} | m/z |
| example | Compound name | Starting amine | Method | (min) | [M + H]⁺ |
| 3R | trans-5-Bromo-2-(1- | trans-4- | 1 | 6.10 | 310.0/ |
| | hydroxycyclohex-4-yl)-2,3- | Aminocyclohexanol | | | 312.0 |
| | dihydroisoindol-1-one | hydrochloride | | | |
| 3S | 5-Bromo-2-(2-hydroxy-5- | 3-Amino-4- | 1 | 6.10 | 380.9/ |
| | sulfamoylphenyl)-2,3- | hydroxybenzenesulfonamide | | | 382.9 |
| | dihydroisoindol-1-one | | | | [M − H]⁻ |
| 3T | 5-Bromo-2-(indazol-6-yl)-2,3- | 6-Aminoindazole | 1 | 7.54 | 327.9/ |
| | dihydroisoindol-1-one | | | | 329.9 |
| 3U | 2-(1-Acetylpiperidin-4-yl)-5- | 1-Acetyl-4- | 1 | 5.91 | 337.0/ |
| | bromo-2,3-dihydroisoindol-1- | aminopiperidine | | | 339.0 |
| | one | | | | |
| 3V | 5-Bromo-2-ethyl-2,3- | Ethylamine | 1 | 6.83 | 240.0/ |
| | dihydroisoindol-1-one | | | | 242.0 |
| 3W | 5-Bromo-2-(2-hydroxy-6- | 2-Amino-3- | 1 | 7.77 | 317.9/ |
| | methylphenyl)-2,3- | methylphenol | | | 319.9 |
| | dihydroisoindol-1-one | | | | |
| 3X | 5-Bromo-2-(thiazol-2-yl)-2,3- | 2-Aminothiazole | 1 | 4.40 | 294.9/ |
| | dihydroisoindol-1-one | | | | 296.9 |
| 3Y | 5-Bromo-2-(4-hydroxyphenyl)- | 4-Aminophenol | 1 | 7.20 | 304.0/ |
| | 2,3-dihydroisoindol-1-one | | | | 306.0 |

### Reference Example 4

### 5-Bromo-2,2-dimethylindan-1-one

To a suspension of sodium hydride (55% in mineral oil, 1.37 g, 31.3 mmol) in toluene (8.5 mL), 5-bromo-1-indanone (3.00 g, 14.2 mmol) and methyl iodide (4.43 g, 31.3 mmol) were added. The mixture was heated at 90° C. overnight and allowed to cool to room temperature. After adding some drops of MeOH to destroy the excess of hydride, EtOAc and water were added. The phases were separated and the aqueous phase was reextracted twice with EtOAc. The combined organic phases were dried over Na₂SO₄ and the solvent was evaporated. The crude product obtained was purified by chromatography on silica gel using hexane-EtOAc mixtures of increasing polarity as eluent, to afford 2.43 g of the title compound (yield: 72%).

¹H NMR (300 MHz, CDCl₃) δ (TMS): 1.25 (s, 6H), 2.98 (s, 2H), 7.51 (d, J=8.4 Hz, 1H), 7.60-7.63 (complex signal, 2H).

### Reference Example 5

### 2,2-Dimethyl-6-methoxy-1,2,3,4-tetrahydronaphthalen-1-one

To a suspension of sodium hydride (55% in mineral oil, 26.80 g, 0.55 mol) in benzene (159 mL), 6-methoxy-1,2,3,4-tetrahydronaphthalen-1-one (50.00 g, 0.28 mol) and methyl iodide (99.10 g, 0.69 mol) were added. The mixture was heated to reflux overnight and allowed to cool to room temperature. After adding some drops of MeOH to destroy the excess of hydride, EtOAc and water were added. The phases were separated and the aqueous phase was reextracted with EtOAc. The combined organic phases were dried over Na₂SO₄ and the solvent was evaporated to afford the title compound (quantitative yield).

¹H NMR (80 MHz, CDCl₃) δ (TMS): 1.19 (s, 6H), 1.94 (t, J=6.5 Hz, 2H), 2.93 (t, J=6.5 Hz, 2H), 3.82 (s, 3H), 6.67 (broad s, 1H), 6.80 (dd, J=9 Hz, J′=2 Hz, 1H), 7.99 (d, J=9 Hz, 1H).

### Reference Example 6

### 2,2-Dimethyl-6-hydroxy-1,2,3,4-tetrahydronaphthalen-1-one

A mixture of 2,2-dimethyl-6-methoxy-1,2,3,4-tetrahydronaphthalen-1-one (20.0 g, 98 mmol, obtained in reference example 5) and 48% aq HBr (279 mL) was heated to reflux for 2 h. Then HBr was distilled off and the reaction crude was allowed to cool to room temperature and diluted with water and ethyl ether. The phases were separated and the product was extracted from the organic phase with 1N NaOH. The basic aqueous phase was acidified with 2N HCl and the solid thus obtained was isolated by filtration and dried under vacuum, to afford 16.06 g of the desired compound as a tan solid (yield: 86%).

¹H NMR (300 MHz, CDCl₃) δ (TMS): 1.21 (s, 6H), 1.96 (t, J=6.3 Hz, 2H), 2.92 (t, J=6.3 Hz, 2H), 5.62 (s, 1H, OH), 6.65 (d, J=2.4 Hz, 1H), 6.76 (dd, J=8.4 Hz, J′=2.4 Hz, 1H), 7.98 (d, J=8.4 Hz, 1H).

### Reference Example 7

### 2,2-Dimethyl-1-oxo-1,2,3,4-tetrahydronaphthalen-6-yl trifluoromethanesulfonate

To a solution of 2,2-dimethyl-6-hydroxy-1,2,3,4-tetrahydronaphthalen-1-one (15.00 g, 78.8 mmol, obtained in reference example 6) in pyridine (40 mL), cooled at 0° C., trifluoromethanesulfonic anhydride (24.46 g, 86.7 mmol) was added. The reaction mixture was allowed to warm to room temperature and stirred overnight. After dilution with water and EtOAc, the phases were separated and the aqueous phase was reextracted 3 times with EtOAc. The combined organic phases were washed with water and twice with 10% HCl, dried over Na₂SO₄ and the solvent was evaporated. The crude product obtained was purified by chromatography on silica gel using hexane-EtOAc mixtures of increasing polarity as eluent, to afford 21.54 g of the desired compound (yield: 85%).

¹H NMR (300 MHz, CDCl₃) δ (TMS): 1.23 (s, 6H), 2.02 (t, J=6.3 Hz, 2H), 3.03 (t, J=6.3 Hz, 2H), 7.15 (d, J=2.4 Hz, 1H), 7.20 (dd, J=8.7 Hz, J′=2.4 Hz, 1H), 8.13 (d, J=8.7 Hz, 1H).

### Reference Example 8

### Ethyl N-[2-(3-methoxyphenyl)ethyl]carbamate

To a solution of 3-methoxyphenetylamine (25.00 g, 0.17 mol) and TEA (25 mL, 0.18 mol) in CH₂Cl₂ (500 mL), cooled at 0° C., ethyl chloroformate (19.53 g, 0.18 mol) was added dropwise and the reaction mixture was stirred at 0° C. for 1.5 h. Water was then added and the phases were separated. The aqueous phase was reextracted with CH₂Cl₂. The combined organic phases were dried over Na₂SO₄ and the solvent was evaporated to afford the desired compound (quantitative yield).

¹H NMR (300 MHz, CDCl₃) δ (TMS): 1.23 (t, J=7.2 Hz, 3H), 2.78 (t, J=6.9 Hz, 2H), 3.43 (q, J=6.6 Hz, 2H), 3.80 (s, 3H), 4.10 (q, J=6.9 Hz, 2H), 4.69 (broad s, 1H), 6.74-6.79 (complex signal, 3H), 7.22 (t, J=7.8 Hz, 1H).

### Reference Example 9

### 6-Methoxy-1,2,3,4-tetrahydroisoquinolin-1-one

A mixture of ethyl N-[2-(3-methoxyphenyl)ethyl]carbamate (18.98 g, 85.0 mmol, obtained in reference example 8) and polyphosphoric acid (60 g) was heated at 120° C. for 3 h and then allowed to cool to 60° C. Water and EtOAc were added and the mixture was allowed to cool to room temperature. The phases were separated and the aqueous phase was reextracted several times with CHCl₃. The combined organic phases were dried over Na₂SO₄ and the solvent was evaporated. The crude product obtained was purified by chromatography on silica gel using EtOAc-MeOH mixtures of increasing polarity as eluent, to afford 10.24 g of the desired compound (yield: 68%).

¹H NMR (300 MHz, CDCl₃) δ (TMS): 2.97 (m, 2H), 3.55 (m, 2H), 3.85 (s, 3H), 6.31 (broad s, 1H), 6.70 (d, J=2.1 Hz, 1H), 6.85 (dd, J=8.7 Hz, J′=2.4 Hz, 1H), 8.01 (d, J=8.4 Hz, 1H).

### Reference Example 10

### 2-(2-Chlorophenyl)-6-methoxy-1,2,3,4-tetrahydroisoquinolin-1-one

To a solution of 6-methoxy-1,2,3,4-tetrahydroisoquinolin-1-one (1.50 g, 8.5 mmol, obtained in reference example 9) in N-methylpyrrolidone (4 mL) under argon, 1-bromo-2-chlorobenzene (2.34 g, 12.3 mmol), copper (I) iodide (0.33 g, 1.7 mmol) and potassium carbonate (2.33 g, 16.9 mmol) were added and the mixture was heated at 200° C. overnight. It was allowed to cool and CHCl₃ and 1N NaOH were added. The phases were separated and the aqueous phase was reextracted 2 times with CHCl₃. The combined organic phases were dried over Na₂SO₄ and the solvent was evaporated. The crude product thus obtained was purified by chromatography on silica gel using hexane-EtOAc mixtures of increasing polarity as eluent, to afford 2.01 g of the desired compound (yield: 77%).

LC-MS (method 1): t_{R}=8.05 min; m/z=288.1/290.1 [M+H]⁺.

### Reference Example 11

### 2-(2-Chlorophenyl)-6-hydroxy-1,2,3,4-tetrahydroisoquinolin-1-one

To a solution of 2-(2-chlorophenyl)-6-methoxy-1,2,3,4-tetrahydroisoquinolin-1-one (2.01 g, 7.0 mmol, obtained in reference example 10) in dry CH₂Cl₂ (40 mL) under argon, cooled at −78° C., boron tribromide (1M in CH₂Cl₂, 13.9 mL, 13.9 mmol) was added. The mixture was allowed to warm to room temperature and stirred overnight. After cooling with ice, 1N HCl was added and the mixture was stirred at 30° C. for 30 min. The phases were then separated and the aqueous phase was reextracted with CHCl₃. The combined organic phases were dried over Na₂SO₄ and the solvent was evaporated to afford 1.86 g of the desired compound (yield: 98%).

LC-MS (method 1): t_{R}=6.41 min; m/z=274.1/276.1 [M+H]⁺.

### Reference Example 12

### 2-(2-Chlorophenyl)-1-oxo-1,2,3,4-tetrahydroisoquinolin-6-yl trifluoromethanesulfonate

To a solution of 2-(2-chlorophenyl)-6-hydroxy-1,2,3,4-tetrahydroisoquinolin-1-one (1.82 g, 6.7 mmol, obtained in reference example 11) in CH₂Cl₂ (50 mL), pyridine (1.1 mL, 13.3 mmol) was added. The solution was cooled at 0° C. and trifluoromethanesulfonic anhydride (2.06 g, 7.3 mmol) was added. The reaction mixture was allowed to warm to room temperature and stirred overnight. After dilution with water, the phases were separated and the aqueous phase was reextracted with CH₂Cl₂. The combined organic phases were washed with 1N HCl, dried over Na₂SO₄ and the solvent was evaporated. The crude product obtained was purified by chromatography on silica gel using hexane-EtOAc mixtures of increasing polarity as eluent, to afford 2.14 g of the desired compound (yield: 80%).

LC-MS (method 1): t_{R}=9.65 min; m/z=406.0/408.0 [M+H]⁺.

### Reference Example 13

### 2-Ethyl-6-methoxy-1,2,3,4-tetrahydroisoquinolin-1-one

To a solution of 6-methoxy-1,2,3,4-tetrahydroisoquinolin-1-one (3.00 g, 16.9 mmol, obtained in reference example 9) in toluene (40 mL) and THF (40 mL), sodium hydride (55% in mineral oil, 3.80 g, 87.2 mmol) was added portionwise. Ethyl iodide (6.73 g, 43.2 mmol) was then added and the mixture was heated at 50° C. overnight. Additional ethyl iodide portions (6.73 g, 43.2 mmol) were added for 3 consecutive days while the mixture was heated at 50° C. The reaction mixture was allowed to cool to room temperature and some drops of MeOH were added to destroy the excess of hydride. It was diluted with EtOAc and water and the phases were separated. The aqueous phase was thoroughly reextracted with EtOAc and the combined organic phases were washed with 2N NaOH and 1N HCl. The organic phase was dried over Na₂SO₄ and the solvent was evaporated. The crude product obtained was purified by chromatography on silica gel using hexane-EtOAc mixtures of increasing polarity as eluent, to afford 1.60 g of the title compound (yield: 46%).

¹H NMR (300 MHz, CDCl₃) δ (TMS): 1.20 (t, J=7.2 Hz, 3H), 2.95 (t, J=6.6 Hz, 2H), 3.51-3.64 (complex signal, 4H), 3.64 (s, 3H), 6.65 (d, J=2.7 Hz, 1H), 6.83 (dd, J=8.7 Hz, J′=2.7 Hz, 1H), 8.02 (d, J=8.7 Hz, 1H).

### Reference Example 14

### 2-Ethyl-6-hydroxy-1,2,3,4-tetrahydroisoquinolin-1-one

Following a similar procedure to that described in reference example 11, but starting from 2-ethyl-6-methoxy-1,2,3,4-tetrahydroisoquinolin-1-one (obtained in reference example 13), the desired compound was obtained.

LC-MS (method 1): t_{R}=4.66 min; m/z=192.1 [M+H]⁺.

### Reference Example 15

### 2-Ethyl-1-oxo-1,2,3,4-tetrahydroisoquinolin-6-yl trifluoromethanesulfonate

Following a similar procedure to that described in reference example 12, but starting from 2-ethyl-6-hydroxy-1,2,3,4-tetrahydroisoquinolin-1-one (obtained in reference example 14), the desired compound was obtained.

LC-MS (method 1): t_{R}=8.44 min; m/z=324.0 [M+H]⁺.

### Reference Example 16

### 2-Benzyl-6-methoxy-1,2,3,4-tetrahydroisoquinolin-1-one

Following a similar procedure to that described in reference example 13, but using benzyl bromide instead of ethyl iodide, the desired compound was obtained.

LC-MS (method 1): t_{R}=8.50 min; m/z=268.0 [M+H]⁺.

### Reference Example 17

### 2-Benzyl-6-hydroxy-1,2,3,4-tetrahydroisoquinolin-1-one

Following a similar procedure to that described in reference example 11, but starting from 2-benzyl-6-methoxy-1,2,3,4-tetrahydroisoquinolin-1-one (obtained in reference example 16), the desired compound was obtained.

LC-MS (method 1): t_{R}=6.53 min; m/z=254.2 [M+H]⁺.

### Reference Example 18

### 2-Benzyl-1-oxo-1,2,3,4-tetrahydroisoquinolin-6-yl trifluoromethanesulfonate

Following a similar procedure to that described in reference example 12, but starting from 2-benzyl-6-hydroxy-1,2,3,4-tetrahydroisoquinolin-1-one (obtained in reference example 17), the desired compound was obtained.

LC-MS (method 1): t_{R}=9.82 min; m/z=386.1 [M+H]⁺.

### Reference Example 19

### 4-Methyl-3-(4,4,5,5-tetramethyl[1,3,2]dioxaborolan-2-yl)benzoic acid

To a solution of 3-iodo-4-methylbenzoic acid (3.71 g, 14.2 mmol) in DMF (130 mL), bis(pinacolato)diboron (7.20 g, 28.4 mmol), [1,1′-bis(diphenylphosphino)ferrocene]dichloro-palladium (II) (1.04 g, 1.28 mmol) and potassium acetate (6.95 g, 70.9 mmol) were added under argon. The mixture was heated at 80° C. overnight and then allowed to cool to room temperature. The solvent was evaporated and the residue was diluted with water and EtOAc. The phases were separated and the aqueous phase was extracted with EtOAc. The combined organic phases were washed twice with 3N HCl and dried over Na₂SO₄. The solvent was evaporated and the crude product thus obtained was purified by chromatography on silica gel using hexane-EtOAc mixtures of increasing polarity as eluent, to afford the title compound impurified with starting bis(pinacolato)diboron. The product was slurried in hexane, filtered and dried under vacuum to afford 2.41 g of pure material (yield: 65%).

¹H NMR (300 MHz, CDCl₃) δ (TMS): 1.36 (s, 12H), 2.61 (s, 3H), 7.25 (d, J=8.1 Hz, 1H), 8.02 (dd, J=8.1 Hz, J′=2.1 Hz, 1H), 8.48 (d, J=2.1 Hz, 1H).

LC-MS (method 1): t_{R}=7.57 min; m/z=261.0 [M−H]⁻.

### Reference Example 19A

### 3-(4,4,5,5-Tetramethyl[1,3,2]dioxaborolan-2-yl)benzoic acid

Following a similar procedure to that described in reference example 19, but starting from 3-iodobenzoic acid, the title compound was obtained.

¹H NMR (300 MHz, CDCl₃) δ (TMS): 1.28 (s, 12H), 7.48 (t, J=7.8 Hz, 1H), 8.03 (m, 1H), 8.19 (m, 1H), 8.55 (s, 1H).

### Reference Example 19B

### 4-Chloro-3-(4,4,5,5-tetramethyl[1,3,2]dioxaborolan-2-yl)benzoic acid

Following a similar procedure to that described in reference example 19, but starting from 4-chloro-3-iodobenzoic acid, the title compound was obtained.

¹H NMR (300 MHz, CDCl₃) δ (TMS): 1.38 (s, 12H), 7.45 (d, J=8.4 Hz, 1H), 8.04 (dd, J=8.4 Hz, J′=2.4 Hz, 1H), 8.41 (d, J=2.1 Hz, 1H).

### Reference Example 19C

### 4-Methoxy-3-(4,4,5,5-tetramethyl[1,3,2]dioxaborolan-2-yl)benzoic acid

Following a similar procedure to that described in reference example 19, but starting from 3-iodo-4-methoxybenzoic acid, the title compound was obtained.

¹H NMR (300 MHz, CDCl₃) δ (TMS): 1.36 (s, 12H), 3.91 (s, 3H), 6.90 (d, J=8.7 Hz, 1H), 8.15 (dd, J=8.7 Hz, J′=2.4 Hz, 1H), 8.41 (d, J=2.4 Hz, 1H).

### Reference Example 20

### 4-Methyl-3-(1-oxo-2-phenyl-2,3-dihydroisoindol-5-yl)benzoic acid

To a suspension of 5-bromo-2-phenyl-2,3-dihydroisoindol-1-one (400 mg, 1.39 mmol, obtained in reference example 3), 4-methyl-3-(4,4,5,5-tetramethyl[1,3,2]dioxaborolan-2-yl)benzoic acid (0.36 g, 1.39 mmol, obtained in reference example 19) and Pd(PPh₃)₄ (0.16 g, 0.14 mmol) in 1,2-dimethoxyethane (20 mL), 1M Na₂CO₃ (12 mL) was added under argon. The mixture was heated at 90° C. for 4 h. It was allowed to cool and 2N NaOH and CHCl₃ were added. The phases were separated and the organic phase was reextracted with 2N NaOH. The combined basic aqueous phases were acidified with 3N HCl and extracted with CHCl₃. The combined organic phases were dried over Na₂SO₄ and the solvent was evaporated. The crude product thus obtained was purified by chromatography on silica gel using hexane-EtOAc mixtures of increasing polarity as eluent, to afford 0.13 g of the title compound (yield: 27%).

LC-MS (method 1): t_{R}=8.41 min; m/z=344.0 [M+H]⁺.

### Reference Examples 20A-20O

Following a similar procedure to that described in reference example 20, but starting from the appropriate compounds in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 20A | 3-(2-Benzyl-1-oxo-2,3- | Reference example | 1 | 7.99 | 356.1 [M − H]⁻ |
| | dihydroisoindol-5-yl)-4- | 3A and reference | | | |
| | methylbenzoic acid | example 19 | | | |
| 20B | 3-[2-(1-Hydroxymethylcyclopentyl)- | Reference example | 1 | 7.18 | 366.1 |
| | 1-oxo-2,3- | 3L and reference | | | |
| | dihydroisoindol-5-yl]-4- | example 19 | | | |
| | methylbenzoic acid | | | | |
| 20C | (1S,2S)-3-[2-(2-Hydroxy-1- | Reference example | 1 | 6.36 | 418.1 |
| | hydroxymethyl-2-phenylethyl)- | 3Q and reference | | | |
| | 1-oxo-2,3-dihydroisoindol-5- | example 19 | | | |
| | yl]-4-methylbenzoic acid | | | | |
| 20D | 3-[2-(2,2-Dimethyl-3- | Reference example | 1 | 6.83 | 354.1 |
| | hydroxypropyl)-1-oxo-2,3- | 3M and reference | | | |
| | dihydroisoindol-5-yl]-4- | example 19 | | | |
| | methylbenzoic acid | | | | |
| 20E | trans-3-[2-(1- | Reference example | 1 | 5.96 | 366.1 |
| | Hydroxycyclohex-4-yl)-1-oxo- | 3R and reference | | | |
| | 2,3-dihydroisoindol-5-yl]-4- | example 19 | | | |
| | methylbenzoic acid | | | | |
| 20F | 3-[2-(2-Hydroxyphenyl)-1-oxo- | Reference example | 1 | 7.35 | 360.1 |
| | 2,3-dihydroisoindol-5-yl]-4- | 3N and reference | | | |
| | methylbenzoic acid | example 19 | | | |
| 20G | 3-[2-(2-Hydroxy-5- | Reference example | 1 | 6.18 | 437.0 [M − H]⁻ |
| | sulfamoylphenyl)-1-oxo-2,3- | 3S and reference | | | |
| | dihydroisoindol-5-yl]-4- | example 19 | | | |
| | methylbenzoic acid | | | | |
| 20H | 3-[2-(3-Hydroxyphenyl)-1-oxo- | Reference example | 1 | 7.42 | 360.1 |
| | 2,3-dihydroisoindol-5-yl]-4- | 3O and reference | | | |
| | methylbenzoic acid | example 19 | | | |
| 20I | 3-[2-(2-Hydroxy-6- | Reference example | 1 | 7.44 | 372.0 [M − H]⁻ |
| | methylphenyl)-1-oxo-2,3- | 3W and reference | | | |
| | dihydroisoindol-5-yl]-4- | example 19 | | | |
| | methylbenzoic acid | | | | |
| 20J | 4-Methyl-3-[1-oxo-2-(thiazol- | Reference example | 1 | 5.04 | 351.0 |
| | 2-yl)-2,3-dihydroisoindol-5- | 3X and reference | | | |
| | yl]benzoic acid | example 19 | | | |
| 20K | 3-[2-(4-Hydroxyphenyl)-1-oxo- | Reference example | 1 | 6.90 | 360.0 |
| | 2,3-dihydroisoindol-5-yl]-4- | 3Y and reference | | | |
| | methylbenzoic acid | example 19 | | | |
| 20L | 4-Chloro-3-[2-(2- | Reference example | 3 | 7.67 | 378.3/ |
| | hydroxyphenyl)-1-oxo-2,3- | 3N and reference | | | 380.3 [M − H]₋ |
| | dihydroisoindol-5-yl]benzoic | example 19B | | | |
| | acid | | | | |
| 20M | 3-[2-(5-Chloro-2- | Reference example | 2 | 5.32 | 394.2/ |
| | hydroxyphenyl)-1-oxo-2,3- | 3AB and reference | | | 396.2 |
| | dihydroisoindol-5-yl]-4- | example 19 | | | |
| | methylbenzoic acid | | | | |
| 20N | 3-[2-(4-Chloro-2- | Reference example | 2 | 5.28 | 392.1/ |
| | hydroxyphenyl)-1-oxo-2,3- | 3AC and reference | | | 394.1 [M − H]₋ |
| | dihydroisoindol-5-yl]-4- | example 19 | | | |
| | methylbenzoic acid | | | | |
| 20O | 3-[2-(2-hydroxyphenyl)-1-oxo- | Reference example | 2 | 4.53 | 376.4 |
| | 2,3-dihydroisoindol-5-yl]-4- | 3N and reference | | | |
| | methoxybenzoic acid | example 19C | | | |

### Reference Examples 21-25

Following a similar procedure to that described in reference example 20, but starting from the appropriate compounds in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 21 | 3-(2,2-Dimethyl-1-oxoindan-5- | Reference example 4 | 1 | 8.37 | 293.1 |
| | yl)-4-methylbenzoic acid | and reference | | | [M − H]⁻ |
| | | example 19 | | | |
| 22 | 3-(2-Ethyl-1-oxo-1,2,3,4- | Reference example | 1 | — | NMR |
| | tetrahydroisoquinolin-6-yl)-4- | 15 and reference | | | See |
| | methylbenzoic acid | example 19 | | | below |
| 23 | 3-(2-Benzyl-1-oxo-1,2,3,4- | Reference example | 1 | 8.63 | 372.1 |
| | tetrahydroisoquinolin-6-yl)-4- | 18 and reference | | | |
| | methylbenzoic acid | example 19 | | | |
| 23A | 3-(2-Benzyl-1-oxo-1,2,3,4- | Reference example | 1 | 8.06 | 358.2 |
| | tetrahydroisoquinolin-6- | 18 and reference | | | |
| | yl)benzoic acid | example 19A | | | |
| 24 | 3-[2-(2-Chlorophenyl)-1-oxo- | Reference example | 1 | 8.48 | 392.1/ |
| | 1,2,3,4-tetrahydroisoquinolin- | 12 and reference | | | 394.1 |
| | 6-yl]-4-methylbenzoic acid | example 19 | | | |
| 25 | 3-(2,2-Dimethyl-1-oxo-1,2,3,4- | Reference example 7 | 1 | 9.56 | 309.2 |
| | tetrahydronaphthalen-6-yl)-4- | and reference | | | |
| | methylbenzoic acid | example 19 | | | |

Reference example 22: ¹H NMR (300 MHz, CDCl₃) δ (TMS): 1.24 (complex signal, 3H), 2.34 (s, 3H), 3.05 (t, J=6.6 Hz, 2H), 3.60-3.71 (complex signal, 4H), 7.14 (broad s, 1H), 7.30 (m, 1H), 7.38 (d, J=8.1 Hz, 1H), 7.96-8.01 (complex signal, 2H), 8.14 (d, J=7.8 Hz, 1H).

### Reference Example 26

### 5-(5-Amino-2-methylphenyl)-2-phenyl-2,3-dihydroisoindol-1-one

To a solution of 4-methyl-3-(1-oxo-2-phenyl-2,3-dihydroisoindol-5-yl)benzoic acid (0.39 g, 1.14 mmol, obtained in reference example 20) in DMF (30 mL), under argon, a solution of TEA (0.17 g, 1.71 mmol) in DMF (3 mL) was added dropwise followed by a solution of diphenyl phosphoryl azide (0.47 g, 1.71 mmol) in DMF (3 mL), and the mixture was stirred at room temperature for 3 h. After adding water (1.6 mL), the reaction mixture was heated at 100° C. for 1 h and then it was allowed to cool to room temperature. The solvent was evaporated and CHCl₃ was added. The organic phase was washed 3 times with saturated NaHCO₃, dried over Na₂SO₄ and the solvent was evaporated. The crude product obtained was purified by chromatography on silica gel using hexane-EtOAc mixtures of increasing polarity as eluent, to afford 0.18 g of the title compound (yield: 50%).

LC-MS (method 1): t_{R}=6.90 min; m/z=315.2 [M+H]⁺.

### Reference Example 27

### 5-(5-Amino-2-methylphenyl)-2,2-dimethylindan-1-one

Following a similar procedure to that described in reference example 26, but starting from 3-(2,2-dimethyl-1-oxoindan-5-yl)-4-methylbenzoic acid (obtained in reference example 21), the desired compound was obtained.

LC-MS (method 1): t_{R}=6.48 min; m/z=266.2 [M+H]⁺.

### Reference Example 28

### N-Cyclopropyl-3-iodo-4-methylbenzamide

To a solution of 3-iodo-4-methylbenzoic acid (4.5 g, 17.2 mmol) in DMF (150 mL), EDC.HCl (3.93 g, 20.5 mmol), HOBT (2.32 g, 17.2 mmol), and N-methylmorpholine (5.21 g, 51.5 mmol)) were added and the mixture was stirred at room temperature for 1 h. Cyclopropylamine (0.98 g, 17.2 mmol) was added and the mixture was stirred at room temperature overnight. The solvent was evaporated and CHCl₃ and saturated NaHCO₃ were added. The phases were separated and the organic phase was then dried over Na₂SO₄. The solvent was evaporated and the crude product obtained was purified by chromatography on silica gel using hexane-EtOAc mixtures of increasing polarity as eluent, to afford 4.12 g of the title compound (yield: 80%).

LC-MS (method 1): t_{R}=7.39 min; m/z=302.0 [M+H]⁺.

### Reference Example 29

### N-Cyclopropyl-4-methyl-3-(4,4,5,5-tetramethyl[1,3,2]dioxaborolan-2-yl)benzamide

Following a similar procedure to that described in reference example 19, but starting from N-cyclopropyl-3-iodo-4-methylbenzamide (obtained in reference example 28), the desired compound was obtained.

LC-MS (method 1): t_{R}=8.58 min; m/z=302.2 [M+H]⁺.

### Reference Example 30

### 4-(3-Aminophenyl)morpholine

### a) 4-(3-Nitrophenyl)morpholine

To a solution of morpholine (6.8 mL, 77.9 mmol) in DMSO (25 mL), 1-fluoro-3-nitrobenzene (2.0 g, 14.2 mmol) was added and the mixture was heated at 110° C. for 48 h. Additional morpholine (3.4 mL, 38.9 mmol) was added and stirring at 110° C. was continued for another 24 h. The reaction mixture was then poured over water, and the precipitate thus obtained was filtered and dried in a vacuum oven to afford 2.35 g of the title compound (yield: 79%).

LC-MS (method 1): t_{R}=7.18 min; m/z=209.1 [M+H]⁺.

### b) Title Compound

To a solution of 4-(3-nitrophenyl)morpholine (2.34 g, 11.3 mmol, obtained in section a) in a 4:1 mixture of EtOH and DMF (120 mL), 0.23 g of 10% Pd on active carbon (wet, 50% water) were added and it was stirred at room temperature under a hydrogen atmosphere for 4 h. The mixture was filtered through a pad of celite and the filtrate was concentrated to dryness to afford 1.87 g of the title compound (yield: 93%).

LC-MS (method 1): t_{R}=1.47 min; m/z=179.2 [M+H]⁺.

### Reference Example 31

### 2-(3-Aminophenyl)pyridine

To a suspension of 2-bromopyridine (0.5 g, 3.2 mmol), 3-aminophenylboronic acid (0.49 g, 3.2 mmol), anhydrous K₂CO₃ (0.87 g, 6.3 mmol) and Pd(PPh₃)₄ (0.36 g, 0.32 mmol) in 1,2-dimethoxyethane (50 mL) under argon, water (0.66 mL) was added. The mixture was heated under argon at 80° C. overnight. It was allowed to cool and water and EtOAc were added. The phases were separated and the aqueous phase was reextracted with EtOAc. The combined organic phases were dried over Na₂SO₄ and the solvent was evaporated. The crude product obtained was purified by chromatography on silica gel using hexane-EtOAc mixtures of increasing polarity as eluent, to afford 0.22 g of the title compound (yield: 42%).

LC-MS (method 1): t_{R}=1.46 min; m/z=171.2 [M+H]⁺.

### Reference Example 32

### 5-(Cyclopropylaminocarbonyl)-3-fluoro-2-methylboronic acid

### a) 3-Fluoro-5-iodo-4-methylbenzoic acid

To a mixture of 3-fluoro-4-methylbenzoic acid (1.54 g, 10.0 mmol) in trifluoromethanesulfonic acid (10 mL), cooled to 0° C., N-iodosuccinimide (2.25 g, 10.0 mmol) was added in portions. The mixture was stirred at 0° C. for 3 h and then at room temperature overnight. The crude was poured over 40 mL of icy water. The solid that precipitated was filtered and washed with water. This crude solid was dissolved in EtOAc and washed with brine. The organic phase was dried over Na₂SO₄ and the solvent was evaporated to afford 2.3 g of the title compound (yield: 82%).

LC-MS (method 2): t_{R}=4.17 min; m/z=279.2 [M−H]⁻.

### b) 3-Fluoro-5-iodo-4-methylbenzoyl chloride

A mixture of 3-fluoro-5-iodo-4-methylbenzoic acid (2.3 g, 8.2 mmol, obtained in section a) in thionyl chloride (3 mL) was heated at 100° C. for 2.5 h. The solvent was distilled off to afford the title compound as a crude product that was directly used in the following step.

### c) N-Cyclopropyl 3-fluoro-5-iodo-4-methylbenzamide

A mixture of 3-fluoro-5-iodo-4-methylbenzoyl chloride (8.2 mmol, obtained in section b), sodium carbonate (2.5 g, 23.5 mmol) and cyclopropylamine (1.3 mL, 18.7 mmol) in CH₂Cl₂ (10 mL) was stirred at room temperature for 72 h. The solid that precipitates is filtered-off and washed with CH₂Cl₂ and EtOAc. The filtrate is concentrated to dryness and the crude product thus obtained was purified by chromatography on silica gel using hexane-EtOAc mixtures of increasing polarity as eluent, to afford 1.8 g of the title compound (yield: 69%).

LC-MS (method 2): t_{R}=7.41 min; m/z=320.3 [M+H]⁺.

### d) Title Compound

A mixture of N-Cyclopropyl 3-fluoro-5-iodo-4-methylbenzamide (1.8 g, 5.6 mmol, obtained in section c) in THF (27 mL) was cooled to 0° C. under an argon atmosphere. Then, sodium hydride (0.44 g 60% in mineral oil, 11 mmol) was added in portions. When hydrogen evolution stopped, the reaction mixture was cooled at −78° C. and n-butyllithium (7.2 mL of a solution 1.6M in hexanes, 11.5 mmol) was slowly added over a period of 25 min maintaining the temperature below −70° C. Then, triisopropyl borate (2.88 mL, 12.4 mmol) was slowly added and the mixture was stirred at −70° C. for further 4 h. Water (7.2 mL) was then added to quench the reaction, and the mixture was allowed to warm to 5° C. EtOAc and saturated ammonium chloride were added and the phases were separated. The organic phase was washed with additional saturated ammonium chloride and brine and dried over Na₂SO₄ and the solvent was evaporated. The crude product obtained was purified by chromatography on silica gel using hexane-EtOAc mixtures of increasing polarity as eluent, to afford 0.62 g of the title compound (yield: 46%).

LC-MS (method 2): t_{R}=4.11 min; m/z=238.4 [M+H]⁺.

### Example 1

### N-Cyclopropyl-4-methyl-3-(1-oxo-2-phenyl-2,3-dihydroisoindol-5-yl)benzamide

To a solution of 4-methyl-3-(1-oxo-2-phenyl-2,3-dihydroisoindol-5-yl)benzoic acid (62 mg, 0.18 mmol, obtained in reference example 20) in DMF (5 mL), cyclopropylamine (12 mg, 0.21 mmol), HOBT (24 mg, 0.18 mmol), PyBOP (94 mg, 0.18 mmol) and N,N-diisopropylethylamine (0.09 mL) were added and the mixture was stirred at room temperature overnight. The solvent was evaporated and CHCl₃ and 1N Na₂CO₃ were added. The phases were separated and the organic phase was dried over Na₂SO₄. The solvent was evaporated and the crude product thus obtained was purified by chromatography on silica gel using hexane-EtOAc mixtures of increasing polarity as eluent, to afford 34 mg of the title compound (yield: 49%).

LC-MS (method 1): t_{R}=8.38 min; m/z=383.0 [M+H]⁺.

### Examples 1A-1C

Following a similar procedure to that described in example 1, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 1A | N-Cyclopropylmethyl-4- | Reference example | 1 | 8.97 | 397.1 |
| | methyl-3-(1-oxo-2-phenyl-2,3- | 20 and | | | |
| | dihydroisoindol-5- | cyclopropylmethylamine | | | |
| | yl)benzamide | | | | |
| 1B | 3-(2-Benzyl-1-oxo-2,3- | Reference example | 1 | 8.11 | 397.2 |
| | dihydroisoindol-5-yl)-N- | 20A and | | | |
| | cyclopropyl-4- | cyclopropylamine | | | |
| | methylbenzamide | | | | |
| 1C | 3-(2-Benzyl-1-oxo-2,3- | Reference example | 1 | 8.66 | 411.1 |
| | dihydroisoindol-5-yl)-N- | 20A and | | | |
| | cyclopropylmethyl-4- | cyclopropylmethylamine | | | |
| | methylbenzamide | | | | |

### Example 1D

### N-Cyclopropyl-3-[2-(2,2-dimethyl-3-hydroxypropyl)-1-oxo-2,3-dihydroisoindol-5-yl]-4-methylbenzamide

To a solution of 3-[2-(2,2-dimethyl-3-hydroxypropyl)-1-oxo-2,3-dihydroisoindol-5-yl]-4-methylbenzoic acid (85 mg, 0.24 mmol, obtained in reference example 20D) in DMF (3 mL), EDC.HCl (50 mg, 0.26 mmol), HOBT (30 mg, 0.24 mmol) and N-methylmorpholine (67 mg, 0.69 mmol)) were added and the mixture was stirred at room temperature for 1 h. Cyclopropylamine (13 mg, 0.24 mmol) was added and the mixture was stirred at room temperature overnight. The solvent was evaporated and CHCl₃ and saturated NaHCO₃ were added. The phases were separated and the organic phase was washed with brine and then dried over Na₂SO₄. The solvent was evaporated and the crude product obtained was purified by chromatography on silica gel using hexane-EtOAc mixtures of increasing polarity as eluent, to afford 49 mg of the title compound (yield: 52%).

LC-MS (method 1): t_{R}=6.89 min; m/z=393.2 [M+H]⁺.

### Examples 1E-1Q

Following a similar procedure to that described in example 1D, but starting from the appropriate compounds, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 1E | N-Cyclopropyl-3-[2-(1- | Reference example | 1 | 7.19 | 405.1 |
| | hydroxymethylcyclopentyl)-1- | 20B and | | | |
| | oxo-2,3-dihydroisoindol-5-yl]- | cyclopropylamine | | | |
| | 4-methylbenzamide | | | | |
| 1F | (1S,2S)—N-Cyclopropyl-3-[2- | Reference example | 1 | 6.46 | 457.1 |
| | (2-hydroxy-1-hydroxymethyl- | 20C and | | | |
| | 2-phenylethyl)-1-oxo-2,3- | cyclopropylamine | | | |
| | dihydroisoindol-5-yl]-4- | | | | |
| | methylbenzamide | | | | |
| 1G | trans-N-Cyclopropyl-3-[2-(1- | Reference example | 1 | 6.08 | 405.1 |
| | hydroxycyclohex-4-yl)-1-oxo- | 20E and | | | |
| | 2,3-dihydroisoindol-5-yl]-4- | cyclopropylamine | | | |
| | methylbenzamide | | | | |
| 1H | N-Cyclopropyl-3-[2-(2- | Reference example | 1 | 7.28 | 397.1 [M − H]⁻ |
| | hydroxyphenyl)-1-oxo-2,3- | 20F and | | | |
| | dihydroisoindol-5-yl]-4- | cyclopropylamine | | | |
| | methylbenzamide | | | | |
| 1I | N-Cyclopropyl-3-[2-(2- | Reference example | 1 | 6.36 | 476.0 [M − H]⁻ |
| | hydroxy-5-sulfamoylphenyl)-1- | 20G and | | | |
| | oxo-2,3-dihydroisoindol-5-yl]- | cyclopropylamine | | | |
| | 4-methylbenzamide | | | | |
| 1J | N-Cyclopropyl-3-[2-(3- | Reference example | 1 | 7.27 | 399.0 |
| | hydroxyphenyl)-1-oxo-2,3- | 20H and | | | |
| | dihydroisoindol-5-yl]-4- | cyclopropylamine | | | |
| | methylbenzamide | | | | |
| 1K | N-Cyclopropyl-3-[2-(2- | Reference example | 1 | 7.53 | 413.1 |
| | hydroxy-6-methylphenyl)-1- | 20I and | | | |
| | oxo-2,3-dihydroisoindol-5-yl]- | cyclopropylamine | | | |
| | 4-methylbenzamide | | | | |
| 1L | N-Cyclopropyl-4-methyl-3-(1- | Reference example | 1 | 5.07 | 390.0 |
| | oxo-2-(thiazol-2-yl)-2,3- | 20J and | | | |
| | dihydroisoindol-5- | cyclopropylamine | | | |
| | yl)benzamide | | | | |
| 1M | N-Cyclopropyl-3-[2-(4- | Reference example | 1 | 7.05 | 399.1 |
| | hydroxyphenyl)-1-oxo-2,3- | 20K and | | | |
| | dihydroisoindol-5-yl]-4- | cyclopropylamine | | | |
| | methylbenzamide | | | | |
| 1N | 4-Chloro-N-cyclopropyl-3-[2- | Reference example | 2 | 6.93 | 419.3/ |
| | (2-hydroxyphenyl)-1-oxo-2,3- | 20L and | | | 421.3 |
| | dihydroisoindol-5- | cyclopropylamine | | | |
| | yl]benzamide | | | | |
| 1O | N-Cyclopropyl-3-[2-(5-chloro- | Reference example | 2 | 7.63 | 433.2/ |
| | 2-hydroxyphenyl)-1-oxo-2,3- | 20M and | | | 435.2 |
| | dihydroisoindol-5-yl]-4- | cyclopropylamine | | | |
| | methylbenzamide | | | | |
| 1P | N-Cyclopropyl-3-[2-(4-chloro- | Reference example | 2 | 7.53 | 433.2/ |
| | 2-hydroxyphenyl)-1-oxo-2,3- | 20N and | | | 435.2 |
| | dihydroisoindol-5-yl]-4- | cyclopropylamine | | | |
| | methylbenzamide | | | | |
| 1Q | N-Cyclopropyl-3-(2-(2- | Reference example | 2 | 6.45 | 415.4 |
| | hydroxyphenyl)-1-oxo-2,3- | 20O and | | | |
| | dihydroisoindol-5-yl)-4- | cyclopropylamine | | | |
| | methoxybenzamide | | | | |

### Example 2

### N-Cyclopropyl-3-(2,2-dimethyl-1-oxoindan-5-yl)-4-methylbenzamide

Following a similar procedure to that described in example 1, but starting from 3-(2,2-dimethyl-1-oxoindan-5-yl)-4-methylbenzoic acid (obtained in reference example 21), the desired compound was obtained.

LC-MS (method 1): t_{R}=8.34 min; m/z=334.2 [M+H]⁺.

### Examples 2A-2I

Following a similar procedure to that described in example 2, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 2A | N-Cyclopropylmethyl-3-(2,2- | Reference example | 1 | 9.04 | 348.2 |
| | dimethyl-1-oxoindan-5-yl)-4- | 21 and | | | |
| | methylbenzamide | cyclopropylmethylamine | | | |
| 2B | N-Butyl-3-(2,2-dimethyl-1- | Reference example | 1 | 9.46 | 350.2 |
| | oxoindan-5-yl)-4- | 21 and butylamine | | | |
| | methylbenzamide | | | | |
| 2C | 3-(2,2-Dimethyl-1-oxoindan-5- | Reference example | 1 | 10.02 | 370.2 |
| | yl)-4-methyl-N- | 21 and aniline | | | |
| | phenylbenzamide | | | | |
| 2D | 3-(2,2-Dimethyl-1-oxoindan-5- | Reference example | 1 | 5.93 | 371.2 |
| | yl)-4-methyl-N-(pyridin-4- | 21 and 4- | | | |
| | yl)benzamide | aminopyridine | | | |
| 2E | 3-(2,2-Dimethyl-1-oxoindan-5- | Reference example | 1 | 9.03 | 336.2 |
| | yl)-N-isopropyl-4- | 21 and | | | |
| | methylbenzamide | isopropylamine | | | |
| 2F | 3-(2,2-Dimethyl-1-oxoindan-5- | Reference example | 1 | 9.59 | 377.2 |
| | yl)-4-methyl-N-(thiazol-2- | 21 and 2- | | | |
| | yl)benzamide | aminothiazole | | | |
| 2G | 3-(2,2-Dimethyl-1-oxoindan-5- | Reference example | 1 | 9.72 | 455.2 |
| | yl)-4-methyl-N-[3-(morpholin- | 21 and reference | | | |
| | 4-yl)phenyl]benzamide | example 30 | | | |
| 2H | 3-(2,2-Dimethyl-1-oxoindan-5- | Reference example | 1 | 9.53 | 447.3 |
| | yl)-4-methyl-N-[3-(pyridin-2- | 21 and reference | | | |
| | yl)phenyl]benzamide | example 31 | | | |
| 2I | N-Benzyl-3-(2,2-dimethyl-1- | Reference example | 1 | 9.59 | 384.2 |
| | oxoindan-5-yl)-4- | 21 and benzylamine | | | |
| | methylbenzamide | | | | |

### Example 3

### N-Cyclopropyl-3-(2-ethyl-1-oxo-1,2,3,4-tetrahydroisoquinolin-6-yl)-4-methylbenzamide

Following a similar procedure to that described in example 1, but starting from 3-(2-ethyl-1-oxo-1,2,3,4-tetrahydroisoquinolin-6-yl)-4-methylbenzoic acid (obtained in reference example 22), the desired compound was obtained.

LC-MS (method 1): t_{R}=7.12 min; m/z=349.2 [M+H]⁺.

### Example 4

### 3-(2-Benzyl-1-oxo-1,2,3,4-tetrahydroisoquinolin-6-yl)-N-cyclopropyl-4-methylbenzamide

Following a similar procedure to that described in example 1, but starting from 3-(2-benzyl-1-oxo-1,2,3,4-tetrahydroisoquinolin-6-yl)-4-methylbenzoic acid (obtained in reference example 23), the desired compound was obtained.

LC-MS (method 1): t_{R}=8.54 min; m/z=411.3 [M+H]⁺.

### Example 4A

Following a similar procedure to that described in example 4, but starting from the appropriate compounds, the compound in the following table was obtained:

| | | | LC-MS | | |
|---|---|---|---|---|---|
| | | | | t_{R} | m/z |
| Example | Compound name | Starting products | Method | (min) | [M + H]⁺ |
| 4A | 3-(2-Benzyl-1-oxo-1,2,3,4- | Reference example | 1 | 9.22 | 425.3 |
| | tetrahydroisoquinolin-6-yl)- | 23 and | | | |
| | N-cyclopropylmethyl-4- | cyclopropylmethylamine | | | |
| | methylbenzamide | | | | |

### Example 5

### 3-[2-(2-Chlorophenyl)-1-oxo-1,2,3,4-tetrahydroisoquinolin-6-yl]-N-cyclopropyl-4-methylbenzamide

Following a similar procedure to that described in example 1, but starting from 3-[2-(2-chlorophenyl)-1-oxo-1,2,3,4-tetrahydroisoquinolin-6-yl]-4-methylbenzoic acid (obtained in reference example 24), the desired compound was obtained.

LC-MS (method 1): t_{R}=8.60 min; m/z=431.1/433.2 [M+H]⁺.

### Example 6

### N-Cyclopropyl-3-(2,2-dimethyl-1-oxo-1,2,3,4-tetrahydronaphthalen-6-yl)-4-methylbenzamide

Following a similar procedure to that described in example 1, but starting from 3-(2,2-dimethyl-1-oxo-1,2,3,4-tetrahydronaphthalen-6-yl)-4-methylbenzoic acid (obtained in reference example 25), the desired compound was obtained.

LC-MS (method 1): t_{R}=9.23 min; m/z=348.2 [M+H]⁺.

### Example 6A

Following a similar procedure to that described in example 6, but starting from the appropriate compounds, the compound in the following table was obtained:

| | | | LC-MS | | |
|---|---|---|---|---|---|
| | | | | t_{R} | m/z |
| Example | Compound name | Starting products | Method | (min) | [M + H]⁺ |
| 6A | N-Cyclopropylmethyl-3-(2,2- | Reference example | 1 | 9.87 | 362.3 |
| | dimethyl-1-oxo-1,2,3,4- | 25 and | | | |
| | tetrahydronaphthalen-6-yl)-4- | cyclopropylmethylamine | | | |
| | methylbenzamide | | | | |

### Example 7

### N-Cyclopropyl-3-[2-(2-hydroxyethyl)-1-oxo-2,3-dihydroisoindol-5-yl]-4-methylbenzamide

To a suspension of 5-bromo-2-(2-hydroxyethyl)-2,3-dihydroisoindol-1-one (150 mg, 0.59 mmol, obtained in reference example 3H), N-cyclopropyl-4-methyl-3-(4,4,5,5-tetramethyl[1,3,2]dioxaborolan-2-yl)benzamide (176 mg, 0.59 mmol, obtained in reference example 29) and Pd(PPh₃)₄ (67 mg, 0.06 mmol) in 1,2-dimethoxyethane (22 mL), 1M Na₂CO₃ (5.2 mL) was added under argon. The mixture was heated at 90° C. overnight and it was allowed to cool to room temperature. Water and EtOAc were added, the phases were separated and the aqueous phase was reextracted with EtOAc. The combined organic phases were washed with brine and dried over Na₂SO₄ and the solvent was evaporated. The crude product thus obtained was purified by chromatography on silica gel using hexane-EtOAc mixtures of increasing polarity as eluent, to afford 56 mg of the title compound (yield: 28%).

LC-MS (method 1): t_{R}=5.41 min; m/z=351.2 M+H]⁺.

### Examples 7A-7P

Following a similar procedure to that described in example 7, but starting from the appropriate compounds, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 7A | N-Cyclopropyl-4-methyl-3-(1- | Reference example | 1 | 4.51 | 398.2 |
| | oxo-2-(pyridin-4-ylmethyl)-2,3- | 3B and reference | | | |
| | dihydroisoindol-5- | example 29 | | | |
| | yl)benzamide | | | | |
| 7B | N-Cyclopropyl-4-methyl-3-[2- | Reference example | 1 | 7.88 | 442.2 |
| | (3-nitrobenzyl)-1-oxo-2,3- | 3C and reference | | | |
| | dihydroisoindol-5- | example 29 | | | |
| | yl]benzamide | | | | |
| 7C | 3-[2-(3-Cyanophenyl)-1-oxo- | Reference example | 1 | 8.34 | 408.3 |
| | 2,3-dihydroisoindol-5-yl]-N- | 3D and reference | | | |
| | cyclopropyl-4- | example 29 | | | |
| | methylbenzamide | | | | |
| 7D | N-Cyclopropyl-4-methyl-3-[2- | Reference example | 1 | 7.98 | 468.3 |
| | (3-(morpholin-4-yl)phenyl)-1- | 3E and reference | | | |
| | oxo-2,3-dihydroisoindol-5- | example 29 | | | |
| | yl]benzamide | | | | |
| 7E | 3-(2-(Biphenyl-3-yl)-1-oxo-2,3- | Reference example | 1 | 10.04 | 459.3 |
| | dihydroisoindol-5-yl)-N- | 3F and reference | | | |
| | cyclopropyl-4- | example 29 | | | |
| | methylbenzamide | | | | |
| 7F | N-Cyclopropyl-3-[2-(3- | Reference example | 1 | 5.54 | 365.2 |
| | hydroxypropyl)-1-oxo-2,3- | 3G and reference | | | |
| | dihydroisoindol-5-yl]-4- | example 29 | | | |
| | methylbenzamide | | | | |
| 7G | N-Cyclopropyl-4-methyl-3-[2- | Reference example 3I | 1 | 4.39 | 420.2 |
| | (2-(morpholin-4-yl)ethyl)-1- | and reference | | | |
| | oxo-2,3-dihydroisoindol-5- | example 29 | | | |
| | yl]benzamide | | | | |
| 7H | N-Cyclopropyl-4-methyl-3-[1- | Reference example | 1 | 4.59 | 412.2 |
| | oxo-2-(2-pyridin-3-ylethyl)-2,3- | 3J and reference | | | |
| | dihydroisoindol-5- | example 29 | | | |
| | yl]benzamide | | | | |
| 7I | N-Cyclopropyl-3-[2-(indazol-6- | Reference example | 1 | 7.29 | 423.1 |
| | yl)-1-oxo-2,3-dihydroisoindol- | 3T and reference | | | |
| | 5-yl]-4-methylbenzamide | example 29 | | | |
| 7J | N-Cyclopropyl-3-[2-(indol-5- | Reference example | 1 | 8.13 | 422.2 |
| | yl)-1-oxo-2,3-dihydroisoindol- | 3K and reference | | | |
| | 5-yl]-4-methylbenzamide | example 29 | | | |
| 7K | 3-[2-(1-Acetylpiperidin-4-yl)-1- | Reference example | 1 | 6.00 | 432.2 |
| | oxo-2,3-dihydroisoindol-5-yl]- | 3U and reference | | | |
| | N-cyclopropyl-4- | example 29 | | | |
| | methylbenzamide | | | | |
| 7L | N-Cyclopropyl-3-[2-(6- | Reference example | 1 | 7.73 | 414.1 |
| | methoxypyridin-3-yl)-1-oxo- | 3P and reference | | | |
| | 2,3-dihydroisoindol-5-yl]-4- | example 29 | | | |
| | methylbenzamide | | | | |
| 7M | N-Cyclopropyl-3-[2-ethyl-1- | Reference example | 1 | 6.82 | 335.1 |
| | oxo-2,3-dihydroisoindol-5-yl]- | 3V and reference | | | |
| | 4-methylbenzamide | example 29 | | | |
| 7N | N-Cyclopropyl-3-[2-(2- | Reference example | 2 | 7.33 | 413.3 |
| | methoxyphenyl)-1-oxo-2,3- | 3AA and reference | | | |
| | dihydroisoindol-5-yl]-4- | example 29 | | | |
| | methylbenzamide | | | | |
| 7O | N-Cyclopropyl-5-fluoro-3-[2- | Reference example | 2 | 7.15 | 415.3 [M − H]⁻ |
| | (2-hydroxyphenyl)-1-oxo-2,3- | 3N and reference | | | |
| | dihydroisoindol-5-yl]-4- | example 32 | | | |
| | methylbenzamide | | | | |
| 7P | N-Cyclopropyl-5-fluoro-3-[2- | Reference example | 2 | 6.65 | 409.4 [M − H]⁻ |
| | (2,2-dimethyl-3- | 3M and reference | | | |
| | hydroxypropyl)-1-oxo-2,3- | example 32 | | | |
| | dihydroisoindol-5-yl]-4- | | | | |
| | methylbenzamide | | | | |

### Example 8

### 2-Cyclopropyl-N-[4-methyl-3-(1-oxo-2-phenyl-2,3-dihydroisoindol-5-yl)phenyl]acetamide

To a solution of 5-(5-amino-2-methylphenyl)-2-phenyl-2,3-dihydroisoindol-1-one (90 mg, 0.28 mmol, obtained in reference example 26) in DMF (8 mL), cyclopropylacetic acid (34 mg, 0.34 mmol), HOBT (38 mg, 0.28 mmol), PyBOP (145 mg, 0.28 mmol) and N,N-diisopropylethylamine (0.15 mL) were added and the mixture was stirred at room temperature overnight. The solvent was evaporated and CHCl₃ and saturated NaHCO₃ were added. The phases were separated and the organic phase was dried over Na₂SO₄. The solvent was evaporated and the crude product thus obtained was purified by chromatography on silica gel using hexane-EtOAc mixtures of increasing polarity as eluent, to afford 70 mg of the title compound (yield: 62%).

LC-MS (method 1): t_{R}=9.76 min; m/z=397.2 [M+H]⁺.

### Examples 8A-8D

Following a similar procedure to that described in example 8, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 8A | N-[4-Methyl-3-(1-oxo-2- | Reference example | 1 | 9.75 | 407.1 |
| | phenyl-2,3-dihydroisoindol-5- | 26 and 3-furoic acid | | | [M − H]⁻ |
| | yl)phenyl]furan-3- | | | | |
| | carboxamide | | | | |
| 8B | N-[3-(2,2-Dimethyl-1- | Reference example | 1 | 9.23 | 334.2 |
| | oxoindan-5-yl)-4- | 27 and cyclopropane | | | |
| | methylphenyl]cyclopropylcarboxamide | carboxylic acid | | | |
| 8C | 2-Cyclopropyl-N-[3-(2,2- | Reference example | 1 | 9.29 | 348.2 |
| | dimethyl-1-oxoindan-5-yl)-4- | 27 and | | | |
| | methylphenyl]acetamide | cyclopropylacetic acid | | | |
| 8D | 2-Chloro-N-[3-(2,2-dimethyl-1- | Reference example | 1 | 9.93 | 405.2/ |
| | oxoindan-5-yl)-4- | 27 and 2- | | | 407.2 |
| | methylphenyl]isonicotinamide | chloroisonicotinic acid | | | |

### Example 9

### N-[3-(2,2-Dimethyl-1-oxoindan-5-yl)-4-methylphenyl]thiophene-3-carboxamide

To a solution of thiophene-3-carboxylic acid (24 mg, 0.19 mmol) in DMF (5 mL), EDC.HCl (43 mg, 0.19 mmol), HOBT (25 mg, 0.19 mmol) and N-methylmorpholine (57 mg, 0.56 mmol)) were added and the mixture was stirred at room temperature for 1 h. 5-(5-Amino-2-methylphenyl)-2,2-dimethylindan-1-one (50 mg, 0.19 mmol, obtained in reference example 27) was added and the mixture was stirred at room temperature overnight. The solvent was evaporated and EtOAc and 1N NaOH were added. The phases were separated and the organic phase was washed with 1N HCl, brine and then dried over Na₂SO₄. The solvent was evaporated and the crude product obtained was purified by chromatography on silica gel using hexane-EtOAc mixtures of increasing polarity as eluent, to afford 46 mg of the title compound (yield: 66%).

LC-MS (method 1): t_{R}=9.70 min; m/z=376.1 [M+H]⁺.

### Example 9A

Following a similar procedure to that described in example 9, but starting from the appropriate compounds, the compound in the following table was obtained:

| | | | LC-MS | | |
|---|---|---|---|---|---|
| | | Starting | | t_{R} | m/z |
| Example | Compound name | products | Method | (min) | [M + H]⁺ |
| 9A | N-[3-(2,2-Dimethyl- | Reference | 1 | 9.27 | 360.2 |
| | 1-oxoindan-5-yl)-4- | example | | | |
| | methylphenyl]furan- | 27 and | | | |
| | 3-carboxamide | 3-furoic | | | |
| | | acid | | | |

### Example 10

### N-[3-(2,2-Dimethyl-1-oxoindan-5-yl)-4-methylphenyl]-2-(pyrrolidin-1-yl)isonicotinamide

A solution of 2-chloro-N-[3-(2,2-dimethyl-1-oxoindan-5-yl)-4-methylphenyl] isonicotinamide (105 mg, 0.26 mmol, obtained in example 8D) in pyrrolidine (0.28 mL) was heated at 80° C. overnight. The solvent was evaporated and water and CHCl₃ were added. The phases were separated and the aqueous phase was reextracted with CHCl₃. The combined organic phases were dried over Na₂SO₄. The solvent was evaporated and the crude product obtained was purified by chromatography on silica gel using hexane-EtOAc mixtures of increasing polarity as eluent, to afford 68 mg of the title compound (yield: 59%).

LC-MS (method 1): t_{R}=6.60 min; m/z=440.3 [M+H]⁺.

### Example 10A

Following a similar procedure to that described in example 10, but starting from the appropriate compounds, the compound in the following table was obtained:

| | | | LC-MS | | |
|---|---|---|---|---|---|
| Exam- | | Starting | | t_{R} | m/z |
| ple | Compound name | products | Method | (min) | [M + H]⁺ |
| 10A | N-[3-(2,2-Dimethyl- | Example | 1 | 8.92 | 456.3 |
| | 1-oxoindan-5-yl)-4- | 8D and | | | |
| | methylphenyl]-2- | morpholine | | | |
| | (morpholin-4- | | | | |
| | yl)isonicotinamide | | | | |

### Example 11

### 1-Benzyl-3-[3-(2,2-dimethyl-1-oxoindan-5-yl)-4-methylphenyl]urea

To a solution of 5-(5-amino-2-methylphenyl)-2,2-dimethylindan-1-one (75 mg, 0.28 mmol, obtained in reference example 27) in DMF (1 mL), benzyl isocyanate (45 mg, 0.34 mmol) was added and the mixture was stirred at room temperature overnight. The solvent was evaporated and the crude product thus obtained was purified by chromatography on silica gel using hexane-EtOAc mixtures of increasing polarity as eluent, to afford 84 mg of the title compound (yield: 56%).

LC-MS (method 1): t_{R}=9.51 min; m/z=399.3 [M+H]⁺.

### Example 1A

Following a similar procedure to that described in example 11, but starting from the appropriate compounds, the compound in the following table was obtained:

| | | | LC-MS | | |
|---|---|---|---|---|---|
| | | Starting | | t_{R} | m/z |
| Example | Compound name | products | Method | (min) | [M + H]⁺ |
| 11A | 1-[3-(2,2-Dimethyl- | Reference | 1 | 8.78 | 351.2 |
| | 1-oxoindan-5-yl)-4- | example | | | |
| | methylphenyl]-3- | 27 and | | | |
| | isopropylurea | isopropyl | | | |
| | | isocyanate | | | |

### Example 12

### 3-[2-(3-Aminobenzyl)-1-oxo-2,3-dihydroisoindol-5-yl]-N-cyclopropyl-4-methylbenzamide

To a solution of N-cyclopropyl-4-methyl-3-[2-(3-nitrobenzyl)-1-oxo-2,3-dihydroisoindol-5-yl]benzamide (73 mg, 0.17 mmol, obtained in example 7B) in EtOH (5.5 mL), tin (II) chloride hydrate (0.19 g, 0.83 mmol) was added and the mixture was heated to reflux for 3 h. It was allowed to cool, the solvent was evaporated and the residue was diluted with EtOAc. The organic phase was washed with saturated NaHCO₃ and brine, and dried over Na₂SO₄. The solvent was evaporated and the crude product thus obtained was purified by chromatography on silica gel using hexane-EtOAc mixtures of increasing polarity as eluent, to afford 54 mg of the title compound (yield: 80%).

LC-MS (method 1): t_{R}=5.62 min; m/z=412.3 [M+H]⁺.

### Example 13

### N-Cyclopropyl-3-[2-(3-methanesulfonylaminobenzyl)-1-oxo-2,3-dihydroisoindol-5-yl]-4-methylbenzamide

To a mixture of 3-[2-(3-aminobenzyl)-1-oxo-2,3-dihydroisoindol-5-yl]-N-cyclopropyl-4-methylbenzamide (44 mg, 0.11 mmol, obtained in example 12), 4-dimethylaminopyridine (0.5 mg, 0.004 mmol) and pyridine (10 mg, 0.13 mmol) in dry CH₂Cl₂ (0.5 mL), a solution of methanesulfonyl chloride (15 mg, 0.13 mmol) in dry CH₂Cl₂ (0.5 mL) was added under argon and the mixture was stirred at room temperature overnight. It was then diluted with CH₂Cl₂ and saturated NaHCO₃ and the phases were separated. The aqueous phase was reextracted with CH₂Cl₂ and the combined organic phases were washed with brine and dried over Na₂SO₄. The solvent was evaporated and the crude product thus obtained was purified by chromatography on silica gel using hexane-EtOAc mixtures of increasing polarity as eluent, to afford 45 mg of the title compound (yield: 86%).

LC-MS (method 1): t_{R}=6.89 min; m/z=490.3 [M+H]⁺.

### Example 14

### 3-(2-Benzyl-1-oxo-1,2,3,4-tetrahydroisoquinolin-6-yl)-N-cyclopropylbenzamide

Following a similar procedure to that described in example 1, but starting from 3-(2-benzyl-1-oxo-1,2,3,4-tetrahydroisoquinolin-6-yl)benzoic acid (obtained in reference example 23A), the desired compound was obtained.

LC-MS (method 1): t_{R}=8.15 min; m/z=397.2 [M+H]⁺.

### Example 15

### Biological Assays

Inhibition of p38α Enzyme Activity (Test 1):

In a final volume of 25 μL, a total of 5 μL of the test product (final concentration, 0.001-10 μM), 5-10 mU of p38α with 0.33 mg/mL of myelin basic protein, Mg²⁺ acetate (10 mM) and [γ³³P-ATP] (100 μM, specific activity 500 cpm/pmol) in buffer Tris 25 mM pH7.5, EGTA 0.02 mM is incubated. The reaction is started by adding Mg²⁺[γ ³³P-ATP]. After incubation for 40 min at room temperature, the reaction is quenched by adding 5 μL of 3% phosphoric acid solution. The reaction mixture (10 μL) is passed through a filter (P30) and washed three times for 5 min with a 75 mM phosphoric acid solution and once with methanol before drying it and counting it, by liquid scintillation.

Inhibition of p38α Enzyme Activity (Test 2):

Compound stocks in 100% DMSO are first diluted in DMSO to a concentration of 1×10⁻³ up to 3.2×10⁻⁸ M and then further diluted in kinase assay buffer (10 mM Tris-HCl, pH 7.2, 10 mM MgCl₂, 0.01% tween 20, 0.05% NaN₃, 1 mM DTT) to a concentration range of 4×10⁻⁵ up to 1.3×10⁻⁹ M. Of each compound solution 5 μL is transferred into a 384-wells black Optiplate (Packard, 6007279), followed by the addition of 5 μL of ATP (Boehringer, 519987), 5 μl of Fluorescein-labeled EGFR peptide substrate and 5 μL of active p38α kinase (GST-tagged fusion protein corresponding to full-length human p38α; expressed in E. coli by Upstate, 14-251), all diluted in kinase assay buffer (see final concentrations in Table 1). The mixture is incubated for 2 hours at room temperature (RT). The reaction is stopped by the addition of 60 μL of IMAP binding reagent, which has been diluted 400-fold in IMAP binding buffer (stock concentration 5 times diluted in Milli Q). After incubation for 30 min at RT, FP is measured on an Analyst™ multimode fluorescence plate reader (Molecular Devices) at excitation wavelength of 485 nm and emission wavelength of 530 nm (1 sec/well).

TABLE 1
assay conditions

| Kinase | Final | | Final | ATP final |
|---|---|---|---|---|
| (from Upstate) | concentration | Substrate | concentration | concentration |
| p38α/SAPK2a, | 0.30 U/mL | LVEPLTPSGEAPNQK-(FI) | 240 nM | 20 μM |
| active | | | | |

Data handling is performed as follows: percentage effects are calculated based on no-p38-enzyme-addition as the maximum inhibitory effect and with p38 enzyme addition as the minimum inhibitory effect. In each experiment, individual compound concentrations are tested in duplicate and percentage effect is calculated for each concentration.

### Inhibition of TNF-α Release Induced by LPS in Human Peripheral Blood Mononuclear Cells (PBMCs):

PBMCs: Heparinized venous blood, obtained from healthy volunteers, is diluted with an equal volume of saline phosphate buffer without calcium or magnesium. Aliquots of 30 mL of the mixture are transferred to 50 mL centrifuge tubes containing 15 mL of Ficoll-Hypaque (1.077 g/mL). The tubes are centrifuged at 1200×g for 20 min at room temperature without braking. Approximately two-thirds of the band of platelets lying above the mononuclear cells is removed with a pipette. The mononuclear cells are carefully transferred to a 50 mL tube, washed twice with saline phosphate buffer, centrifuged at 300×g for 10 min at room temperature and resuspended in RPMI supplemented with 1% inactivated fetal bovine serum at a cell density of 2×10⁶ cells/mL.

Assay: 100 μL of mononuclear cells (2×10⁶ cells/mL) are incubated in 96-well plates with 50 μL of the test product (final concentration, 0.001-10 μM) and 50 μL LPS (E. coli 055B5, Sigma) at a final concentration of 400 ng/mL for 19 h at 37° C. in an atmosphere with CO₂ at 5%. The amount of TNFα released in the supernatant is quantified using a commercial ELISA kit (Biosource International).

Compounds of all examples exhibited more than 50% inhibition at 10 μM in at least one of the above assays.

1. A compound of general formula I 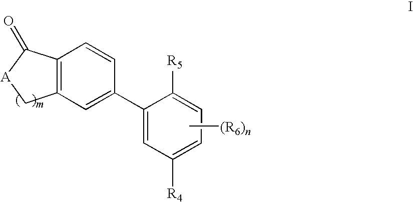
wherein:
A represents CR₁R₂ or NR₃;
and R₂ independently represent C₁₋₄ alkyl;
represents —(CH₂)ₚ—Cy¹, or C₁₋₆ alkyl optionally substituted with one or more R₇;
m represents 1 or 2;
R₄ represents —B—R₈;
R₅ represents hydrogen, C₁₋₄ alkyl, halogen or C₁₋₄ alkoxy;
R₆ can be attached to any available carbon atom of the phenyl ring and represents halogen or methyl;
n represents 0 or 1;
B represents —CONR₉—, —NR₉CO— or —NR₉CONR₉—;
R₇ represents hydroxy, C₁₋₄ alkoxy, halogen, —NR₁₀R₁₀ or phenyl optionally substituted with one or more groups selected from C₁₋₄ alkyl, halogen, C₁₋₄ alkoxy, C₁₋₄ haloalkyl and C₁₋₄ haloalkoxy, and additionally two R₇ groups on the same carbon atom can be bonded together to form a —(CH₂)_{q}— group;
R₈ represents C₁₋₆ alkyl or —(CH₂)ₚ—Cy²;
p represents 0, 1 or 2;
q represents 2, 3, 4, 5 or 6;
Cy¹ represents phenyl, heteroaryl, C₃₋₇ cycloalkyl or heterocyclyl, which can all be optionally substituted with one or more R₁₁;
Cy² represents phenyl, heteroaryl or C₃₋₇ cycloalkyl, which can all be optionally substituted with one or more R₁₂;
R₉ and R₁₀ independently represent hydrogen or C₁₋₄ alkyl;
R₁₁ represents halogen, R₁₃, —OR₁₃, —NO₂, —CN, —COR_{13′}, —CO₂R_{13′}, —CONR_{14′}R_{14′}, —NR_{14′}R_{14′}, —NR_{14′}COR_{13′}, —NR_{14′}CONR_{14′}R_{14′}, —NR_{14′}CO₂R₁₃, NR_{14′}SO₂R₁₃, —SR_{13′}, —SOR₁₃, —SO₂R₁₃, —SO₂NR_{14′}R_{14′}, or Cy³;
R₁₂ represents C₁₋₄ alkyl, halogen, C₁₋₄ alkoxy, C₁₋₄ haloalkyl, C₁₋₄ haloalkoxy, or Cy³;
R₁₃ represents C₁₋₄ alkyl, C₁₋₄ haloalkyl or C₁₋₄ hydroxyalkyl;
R_{13′} represents hydrogen or R₁₃;
R₁₄ represents C₁₋₄ alkyl or C₁₋₄ hydroxyalkyl;
R_{14′} represents hydrogen or R₁₄; and
Cy³ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl and C₁₋₄ haloalkoxy;
or a salt thereof.

2. A compound according to claim 1 wherein R₃ represents —(CH₂)ₚ—Cy¹.

3. A compound according to claim 1 wherein A represents CR₁R₂.

4. A compound according to claim 1 wherein A represents NR₃.

5. A compound according to claim 1 wherein m represents 1.

6. A compound according to claim 1 wherein m represents 2.

7. A compound according to claim 1 wherein R₃ represents —(CH₂)ₚ—Cy¹, p in R₃ is 0 and Cy¹ represents phenyl or heteroaryl, which can all be optionally substituted with one or more R₁₁.

8. A compound according to claim 1 wherein R₅ represents C₁₋₄ alkyl, halogen or C₁₋₄ alkoxy.

9. A compound according to claim 1 wherein B represents —CONR₉—.

10. A compound according to claim 1 wherein R represents —(CH₂)ₚ—Cy².

11. A compound according to claim 10 wherein R₈ represents —(CH₂)ₚ—Cy² and Cy² represents C₃₋₇ cycloalkyl.

12. A compound according to claim 1 selected from:
N-Cyclopropyl-4-methyl-3-(1-oxo-2-phenyl-2,3-dihydroisoindol-5-yl)benzamide;
N-Cyclopropylmethyl-4-methyl-3-(1-oxo-2-phenyl-2,3-dihydroisoindol-5-yl)benzamide;
3-(2-Benzyl-1-oxo-2,3-dihydroisoindol-5-yl)-N-cyclopropyl-4-methylbenzamide;
3-(2-Benzyl-1-oxo-2,3-dihydroisoindol-5-yl)-N-cyclopropylmethyl-4-methylbenzamide;
N-Cyclopropyl-3-[2-(2,2-dimethyl-3-hydroxypropyl)-1-oxo-2,3-dihydroisoindol-5-yl]-4-methylbenzamide;
N-Cyclopropyl-3-[2-(1-hydroxymethylcyclopentyl)-1-oxo-2,3-dihydroisoindol-5-yl]-4-methylbenzamide;
(1S,2S)—N-Cyclopropyl-3-[2-(2-hydroxy-1-hydroxymethyl-2-phenylethyl)-1-oxo-2,3-dihydroisoindol-5-yl]-4-methylbenzamide;
trans-N-Cyclopropyl-3-[2-(1-hydroxycyclohex-4-yl)-1-oxo-2,3-dihydroisoindol-5-yl]-4-methylbenzamide;
N-Cyclopropyl-3-[2-(2-hydroxyphenyl)-1-oxo-2,3-dihydroisoindol-5-yl]-4-methylbenzamide;
N-Cyclopropyl-3-[2-(2-hydroxy-5-sulfamoylphenyl)-1-oxo-2,3-dihydroisoindol-5-yl]-4-methylbenzamide;
N-Cyclopropyl-3-[2-(3-hydroxyphenyl)-1-oxo-2,3-dihydroisoindol-5-yl]-4-methylbenzamide;
N-Cyclopropyl-3-[2-(2-hydroxy-6-methylphenyl)-1-oxo-2,3-dihydroisoindol-5-yl]-4-methylbenzamide;
N-Cyclopropyl-4-methyl-3-(1-oxo-2-(thiazol-2-yl)-2,3-dihydroisoindol-5-yl)benzamide;
N-Cyclopropyl-3-[2-(4-hydroxyphenyl)-1-oxo-2,3-dihydroisoindol-5-yl]-4-methylbenzamide;
4-Chloro-N-cyclopropyl-3-[2-(2-hydroxyphenyl)-1-oxo-2,3-dihydroisoindol-5-yl]benzamide;
N-Cyclopropyl-3-[2-(5-chloro-2-hydroxyphenyl)-1-oxo-2,3-dihydroisoindol-5-yl]-4-methylbenzamide;
N-Cyclopropyl-3-[2-(4-chloro-2-hydroxyphenyl)-1-oxo-2,3-dihydroisoindol-5-yl]-4-methylbenzamide;
N-Cyclopropyl-3-(2-(2-hydroxyphenyl)-1-oxo-2,3-dihydroisoindol-5-yl)-4-methoxybenzamide;
N-Cyclopropyl-3-(2,2-dimethyl-1-oxoindan-5-yl)-4-methylbenzamide;
N-Cyclopropylmethyl-3-(2,2-dimethyl-1-oxoindan-5-yl)-4-methylbenzamide;
N-Butyl-3-(2,2-dimethyl-1-oxoindan-5-yl)-4-methylbenzamide;
3-(2,2-Dimethyl-1-oxoindan-5-yl)-4-methyl-N-phenylbenzamide;
3-(2,2-Dimethyl-1-oxoindan-5-yl)-4-methyl-N-(pyridin-4-yl)benzamide;
3-(2,2-Dimethyl-1-oxoindan-5-yl)-N-isopropyl-4-methylbenzamide;
3-(2,2-Dimethyl-1-oxoindan-5-yl)-4-methyl-N-(thiazol-2-yl)benzamide;
3-(2,2-Dimethyl-1-oxoindan-5-yl)-4-methyl-N-[3-(morpholin-4-yl)phenyl]benzamide;
3-(2,2-Dimethyl-1-oxoindan-5-yl)-4-methyl-N-[3-(pyridin-2-yl)phenyl]benzamide;
N-Benzyl-3-(2,2-dimethyl-1-oxoindan-5-yl)-4-methylbenzamide;
N-Cyclopropyl-3-(2-ethyl-1-oxo-1,2,3,4-tetrahydroisoquinolin-6-yl)-4-methylbenzamide;
3-(2-Benzyl-1-oxo-1,2,3,4-tetrahydroisoquinolin-6-yl)-N-cyclopropyl-4-methylbenzamide;
3-(2-Benzyl-1-oxo-1,2,3,4-tetrahydroisoquinolin-6-yl)-N-cyclopropylmethyl-4-methylbenzamide;
3-[2-(2-Chlorophenyl)-1-oxo-1,2,3,4-tetrahydroisoquinolin-6-yl]-N-cyclopropyl-4-methylbenzamide;
N-Cyclopropyl-3-(2,2-dimethyl-1-oxo-1,2,3,4-tetrahydronaphthalen-6-yl)-4-methylbenzamide;
N-Cyclopropylmethyl-3-(2,2-dimethyl-1-oxo-1,2,3,4-tetrahydronaphthalen-6-yl)-4-methylbenzamide;
N-Cyclopropyl-3-[2-(2-hydroxyethyl)-1-oxo-2,3-dihydroisoindol-5-yl]-4-methylbenzamide;
N-Cyclopropyl-4-methyl-3-(1-oxo-2-(pyridin-4-ylmethyl)-2,3-dihydroisoindol-5-yl)benzamide;
N-Cyclopropyl-4-methyl-3-[2-(3-nitrobenzyl)-1-oxo-2,3-dihydroisoindol-5-yl]benzamide;
3-[2-(3-Cyanophenyl)-1-oxo-2,3-dihydroisoindol-5-yl]-N-cyclopropyl-4-methylbenzamide;
N-Cyclopropyl-4-methyl-3-[2-(3-(morpholin-4-yl)phenyl)-1-oxo-2,3-dihydroisoindol-5-yl]benzamide;
3-(2-(Biphenyl-3-yl)-1-oxo-2,3-dihydroisoindol-5-yl)-N-cyclopropyl-4-methylbenzamide;
N-Cyclopropyl-3-[2-(3-hydroxypropyl)-1-oxo-2,3-dihydroisoindol-5-yl]-4-methylbenzamide;
N-Cyclopropyl-4-methyl-3-[2-(2-(morpholin-4-yl)ethyl)-1-oxo-2,3-dihydroisoindol-5-yl]benzamide;
N-Cyclopropyl-4-methyl-3-[1-oxo-2-(2-pyridin-3-ylethyl)-2,3-dihydroisoindol-5-yl]benzamide;
N-Cyclopropyl-3-[2-(indazol-6-yl)-1-oxo-2,3-dihydroisoindol-5-yl]-4-methylbenzamide;
N-Cyclopropyl-3-[2-(indol-5-yl)-1-oxo-2,3-dihydroisoindol-5-yl]-4-methylbenzamide;
3-[2-(1-Acetylpiperidin-4-yl)-1-oxo-2,3-dihydroisoindol-5-yl]-N-cyclopropyl-4-methylbenzamide;
N-Cyclopropyl-3-[2-(6-methoxypyridin-3-yl)-1-oxo-2,3-dihydroisoindol-5-yl]-4-methylbenzamide;
N-Cyclopropyl-3-[2-ethyl-1-oxo-2,3-dihydroisoindol-5-yl]-4-methylbenzamide;
N-Cyclopropyl-3-[2-(2-methoxyphenyl)-1-oxo-2,3-dihydroisoindol-5-yl]-4-methylbenzamide;
N-Cyclopropyl-5-fluoro-3-[2-(2-hydroxyphenyl)-1-oxo-2,3-dihydroisoindol-5-yl]-4-methylbenzamide;
N-Cyclopropyl-5-fluoro-3-[2-(2,2-dimethyl-3-hydroxypropyl)-1-oxo-2,3-dihydroisoindol-5-yl]-4-methylbenzamide;
2-Cyclopropyl-N-[4-methyl-3-(1-oxo-2-phenyl-2,3-dihydroisoindol-5-yl)phenyl]acetamide;
N-[4-Methyl-3-(1-oxo-2-phenyl-2,3-dihydroisoindol-5-yl)phenyl]furan-3-carboxamide;
N-[3-(2,2-Dimethyl-1-oxoindan-5-yl)-4-methylphenyl]cyclopropylcarboxamide;
2-Cyclopropyl-N-[3-(2,2-dimethyl-1-oxoindan-5-yl)-4-methylphenyl]acetamide;
2-Chloro-N-[3-(2,2-dimethyl-1-oxoindan-5-yl)-4-methylphenyl]isonicotinamide;
N-[3-(2,2-Dimethyl-1-oxoindan-5-yl)-4-methylphenyl]thiophene-3-carboxamide;
N-[3-(2,2-Dimethyl-1-oxoindan-5-yl)-4-methylphenyl]furan-3-carboxamide;
N-[3-(2,2-Dimethyl-1-oxoindan-5-yl)-4-methylphenyl]-2-(pyrrolidin-1-yl)isonicotinamide;
N-[3-(2,2-Dimethyl-1-oxoindan-5-yl)-4-methylphenyl]-2-(morpholin-4-yl)isonicotinamide;
1-Benzyl-3-[3-(2,2-dimethyl-1-oxoindan-5-yl)-4-methylphenyl]urea;
1-[3-(2,2-Dimethyl-1-oxoindan-5-yl)-4-methylphenyl]-3-isopropylurea;
3-[2-(3-Aminobenzyl)-1-oxo-2,3-dihydroisoindol-5-yl]-N-cyclopropyl-4-methylbenzamide;
N-Cyclopropyl-3-[2-(3-methanesulfonylaminobenzyl)-1-oxo-2,3-dihydroisoindol-5-yl]-4-methylbenzamide; and
3-(2-Benzyl-1-oxo-1,2,3,4-tetrahydroisoquinolin-6-yl)-N-cyclopropylbenzamide.

13. A pharmaceutical composition which comprises a compound of formula I according to claim 1 or a pharmaceutically acceptable salt thereof and one or more pharmaceutically acceptable excipients.

14. A method of treating or preventing a disease mediated by p38 in a subject in need thereof which comprises administering to said subject an effective amount of a compound of formula I according to claim 1 or a pharmaceutically acceptable salt thereof.

15. A method according to claim 14, wherein the disease mediated by p38 is selected from immune, autoimmune and inflammatory diseases, cardiovascular diseases, infectious diseases, bone resorption diseases, neurodegenerative diseases, proliferative diseases and processes associated with the induction of cyclooxygenase-2.
